(19) United States
(12) Patent Application Publication
(10) Pub. No.: US 2019/0374221 A1
(43) Pub. Date: Dec. 12, 2019

(54) SUTURE CLIP AND APPLIER TOOL

(71) Applicant: Asfora IP, LLC, Sioux Falls, SD (US)

(72) Inventors: Wilson Theophilo Asfora, Sioux Falls, SD (US); Michael Edward Villalta, Yerington, NV (US)

(21) Appl. No.: 16/437,824

(22) Filed: Jun. 11, 2019

Related U.S. Application Data
(60) Provisional application No. 62/683,504, filed on Jun. 11, 2018.
(60) Provisional application No. 62/730,969, filed on Sep. 13, 2018.
(60) Provisional application No. 62/743,336, filed on Oct. 9, 2018.

(51) Int. Cl. A61B 17/04; A61L 31/02

(52) U.S. Cl. CPC A61B 17/0487; A61B 17/10; A61B 2017/0488; A61L 31/022

(57) ABSTRACT

The disclosure is related to a suture clip for joining two or more portions of tissue (e.g., when closing a surgical incision) and tools for applying the same. The suture clip includes a spring element, two side portions connected to the spring element for actuating the clip into an open configuration, and a clamp portion, which includes opposing closure elements with respective clamping surfaces and at least one spike operatively arranged in relation to one clamping surface such that it extends (perpendicularly or at an angle) toward the opposite clamping surface.

### CROSS-REFERENCE TO RELATED APPLICATIONS

The present application claims priority to U.S. provisional application No. 62/683,504, filed Jun. 11, 2018; U.S. provisional application No. 62/730,969, filed Sep. 13, 2018; and U.S. provisional application No. 62/743,336, filed Oct. 9, 2018, each of which is entitled, “Suture Clip and Applier Tool,” and each of which is hereby incorporated by reference in its entirety.

### TECHNICAL FIELD

The present disclosure pertains to medical devices and procedures for using the same, and more specifically to a suture clip for use in tissue repair.

### BACKGROUND

Various types of sutures, clips and staples are used for closing wounds or joining tissue together to facilitate the healing of the tissue. In certain procedures, such as when closing dural or vascular incisions, increased precision and manipulability may be required to properly place and secure a suture while controlling the tissue and thus improved sutures and procedures for applying the same may be desired to speed the healing process. Thus medical device manufacturer and clinicians continue to seek improvements in the field of surgical clips and sutures.

### SUMMARY

Embodiments disclosed herein generally relate to a suture clip which includes a spring element configured to provide a clamping force urging the clip toward a closed configuration, a pair of opposing side portions connected to the spring element such that a manipulation of the side portions toward one another applies a force against the clamping force of the spring element, and a pair of opposing closure elements coupled to the spring element such that the closure elements are urged toward one another by the clamping force of the spring element. Each of the closure elements includes a clamping surface and a spike extending from the clamping surface arranged such that the clamping surfaces of the opposing closure elements are opposite one another such that they can apply the clamping force to soft tissue positioned between the opposing closure elements and such that the spikes are configured to penetrate the tissue sufficiently to prevent movement of the clamping surface relative to the tissue.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments, wherein identical reference numerals refer to identical or similar elements or features in different views or embodiments shown in the drawings;

FIG. 1 is a view of a suture clip in accordance with some embodiments of the present disclosure;

FIG. 2 is another view of the suture clip of FIG. 1;

FIG. 3 is yet another view of the suture clip of FIG. 1;

FIG. 4 is a view of a suture clip in accordance with further embodiments of the present disclosure;

FIG. 5 is another view of the suture clip of FIG. 4;

FIG. 6 is a view of a suture clip and a method of applying the suture clip in accordance with yet further embodiments of the present disclosure;

FIG. 7 is another view of the suture clip of FIG. 6;

FIG. 8 shows the suture clip of FIG. 6 as it is being applied to bodily tissue to close a wound;

FIG. 9 shows the suture clip of FIG. 6 applied to the bodily tissue to close a wound;

FIG. 10 shows a transverse cross-sectional view of the suture clip and bodily tissue in FIG. 9;

FIGS. 11A and 11B show isometric views of a lobed suture clip in accordance with some embodiments of the present disclosure;

FIG. 12 shows orthographic projection views of the lobed suture clip of FIGS. 11A and 11B;

FIG. 13 shows a strip of material prior to forming it into the lobed suture clip in FIGS. 11A and 11B;

FIG. 14 shows the suture clip of FIGS. 11A and 11B with the clip opened for applying the clip to bodily tissue;

FIG. 15 shows the suture clip of FIG. 14 applied to bodily tissue;

FIG. 16 shows an isometric view of a lobed suture clip in accordance with further embodiments of the present disclosure;

FIG. 17 shows a view of an applier for suture clips in accordance with embodiments of the present disclosure;

FIG. 18 shows a portion of the applier of FIG. 17;

FIG. 19 shows another partial view of the applier of FIG. 17;

FIGS. 20A and 20B shows further partial views of an applier with a suture clip positioned in the applier to illustrate operation thereof;

FIGS. 21A and 21B illustrate further features of an applier tool according to the present disclosure;

FIG. 22 shows an isometric view of a portion of an applier tool according to further examples herein;

FIG. 23 shows a side view of the portion of the applier in FIG. 22;

FIG. 24 shows a top view of the portion of the applier in FIG. 22;

FIG. 25 shows a cross-sectional view of the applier tool in FIG. 22;

FIGS. 26A and 26B show another example of an applier tool in accordance with the present disclosure;

FIG. 27 shows an isometric view of a suture clip according to further examples of the present disclosure;

FIG. 28 shows a bottom view of the clamp portion of the suture clip in FIG. 27 in a closed configuration;

FIG. 29 shows a cross-sectional view of the clamp portion of the suture clip in FIG. 28 taken at line 29-29 in FIG. 28;

FIGS. 30A-30E show isometric and orthographic projection views of another example of a suture clip according to the present disclosure;

FIGS. 31A-31E show isometric and orthographic projection views of yet another example of a suture clip according to present disclosure;

FIGS. 32A-32E show isometric and orthographic projection views of a further example of a suture clip according to the present disclosure;

FIGS. 33A-33E show isometric and orthographic projection views of yet another example of a suture clip according to the present disclosure;

FIGS. 34A-34E show isometric and orthographic projection views of another example of a suture clip according to the present disclosure;

FIG. 35 shows an example of a v-shaped suture clip according to the present disclosure; and

FIG. 36 shows another example of a v-shaped suture clip according to the present disclosure.

### DETAILED DESCRIPTION

Examples described herein generally relate to a suture clip that is configured to hold two or more tissue portions together, such as in the case of holding two sides of an incision closed to promote the healing of tissue and the natural closure of the incision. Tools and procedures for applying a suture clip according to the present disclosure are also described. In some examples, the suture clip is made from a surgical-grade metal, such as 316 stainless steel or titanium, and preferably from a shape-memory alloy (e.g., nickel titanium, also known under the brand name NITINOL).

As will be further described, a suture clip according to the present disclosure may include a spring element and a clamp portion, wherein the spring element is operatively connected to the clamp portion to apply a biasing force urging the clamp portion closed, and wherein the clamp portion includes a pair of opposing surfaces configured to transfer the biasing force to soft tissue to clamp the soft tissue and at least one spike configured to at least partially penetrate the soft tissue to gain purchase on the soft tissue while clamping the soft tissue.

A suture clip according to some examples herein may include a spring element, two opposing side portion connected to the spring element, and a clamp portion connected to the side portions to allow the clamp portion to be opened and closed. When the clamp portion of the suture clip is opened, the clip may be referred to as being in an open configuration. When the clamp portion is closed the clip may be referred to as being in a closed configuration. The spring element is configured to provide a biasing force (also referred to as clamping force) that urges the clip toward the closed configuration. The opposing side portions are connected to the spring element such that a manipulating the side portions toward one another (e.g., squeezing the side portions together) applies a force against the biasing force of the spring element.

In some embodiments, the clamp portion is implemented by two opposing closure elements coupled to the spring element such that the closure elements are urged toward one another by the clamping force of the spring element. In some examples, the closure elements are coupled to the spring element via the side portions. In some such examples, respective ones of the side portions are between respective ones of the closure elements and the spring element. Each of the closure elements includes a clamping surface which, in use, contacts the soft tissue positioned between the closure elements (e.g., opposing tissue on each side of an incision to be held closed for healing) and thereby transfers the clamping force of the spring to the tissue. The closure elements with their respective clamping surfaces may have any suitable geometry (e.g., generally rectangular, circular, elliptical, or any other suitable regular or irregular shape) to transfer sufficient amount of clamping force to the tissue. The closure elements may be configured to provide a clamping footprint, which may be larger than the actual surface that contacts the tissue, such that the clamping force is applied along a desire length and/or locations along the incision. In some examples, the clamp portion may have a clamping footprint that applies a clamping force along, at least a 4cm of the incision. In this manner, the suture clip may promote healing of an incision better than conventional sutures by virtue of holding a larger surface area of the tissue together as compare to conventional filament sutures.

In preferred embodiments, the clamp portions may include one or more traction element(s), such as one or more spikes, which may be provided on at least one or both of the closure elements of jaws. The traction elements may be piercing (such as a spike, spear head, blade, etc.), or non-piercing (such as a toothed configuration clamp portion). In some embodiments, each of the closure elements may include at least one spike extending (e.g., perpendicularly or at an angle) from the respective clamping surface toward the region between the two clamping surfaces. The spikes may extend from any suitable surface of the closure elements as long as the spikes protrude in relation to the clamping surface and extend toward the opposing clamping surface. The spikes may have any suitable geometry or arrangement in relation to the clamping surface to enable the clamp to gain purchase on the tissue being clamped. That is, the one or more spikes (of any suitable geometry, but typically with a pointed tip) may be provided at one or more locations relative to the clamping surfaces such that the spikes sufficiently penetrate the tissue to substantially prevent relative movement between the clamping surface and the tissue. The geometry, number and arrangement of spikes relative to the clamping surface may depend upon the particular application for a given embodiment of the suture clip. For example, a different number, configuration (e.g., length, width, sharpness, etc.) and/or arrangement of spikes may be used for suturing dural tissue, which may be more delicate, versus clips designed for use with muscular tissue, such as in vascular applications. In some embodiments, the spikes may fully penetrate the tissue in order to provide a securing (e.g., non-slip) function to the clip. In some embodiments, the spikes may only partially penetrate through the tissue. In some embodiments, the spikes may be arranged such that the clip applies a clamping force at a location deeper into the tissue (in relation to the edge of the incision) than the location of penetration(s), which may prevent or reduce the risk of fluid leakage through penetrations, if any, created by the spikes. As will be appreciated, the suture clips describe herein may be particularly well suited for applications, such as for dural incision closure, where fluid leakage and/or fluid pressure causing the incision to open may be of greater concern, since the examples herein may provide the ability to apply greater force to hold the incision closed and thus reduce fluid leakage or the risk of the incision opening as compared to conventional suturing techniques or conventional suture clips. For example, with conventional sutures, the dural tissue may become lacerated at the incision holes, which may cause cerebrospinal fluid (CSF) leakage. Thus, in accordance with the principles of the present disclosure, an improved non-penetrative technique for suturing the dura may be by to utilize a suture clip according to the present disclosure, which does not fully penetrate the dural tissue, additionally or optionally applies a clamping force between any penetrations of the dural tissue, and/or only penetrates at fewer locations and/or with a smaller diameter penetration hole thus reducing the risk of CSF leakage. Also, while the clips in the examples herein include spikes, in some embodiments, the spikes may be designed only to grab onto the outer layer of tissue (i.e. to gain traction on the tissue to prevent slippage) without necessarily fully penetrating the tissue, which may be a preferred when suturing in proximity to spinal or brain tissue.

In some embodiments, the suture clip may include traction elements such as spikes, which may be configured to penetrate the tissue (e.g., through the sides of the tissue at the incision, which is being held together by the clip). In some embodiments, the spikes may not penetrate fully but only sufficiently to gain purchase on the tissue and prevent or reduce movement of the tissue relative to the clamp portion, thus ensuring that the two sides of the incision are immobilized from movement relative to one another and thereby promoting the healing process. Other types of traction elements that penetrate the tissue, such as spear head shaped, blade-shaped, or others, may be used in some embodiments. Different types of traction elements may be combined in a single embodiment. Embodiments of a suture clip described herein thus provide both a clamping function and a suturing function, which may be superior to conventional sutures (e.g., filament-type sutures) by increasing the amount of tissue held firmly together which may speed up the healing process. In yet other embodiments, traction elements may be used which do not necessarily penetrate the tissue but nonetheless gain purchase on the tissue to prevent or minimize slippage while of the clamp.

Referring now to the figures, specific examples of suture clips according to the present disclosure will be described. It will be understood that these examples are provided for illustration only and other variations are envisioned, such as combining elements from different examples, or eliminating components from any given example.

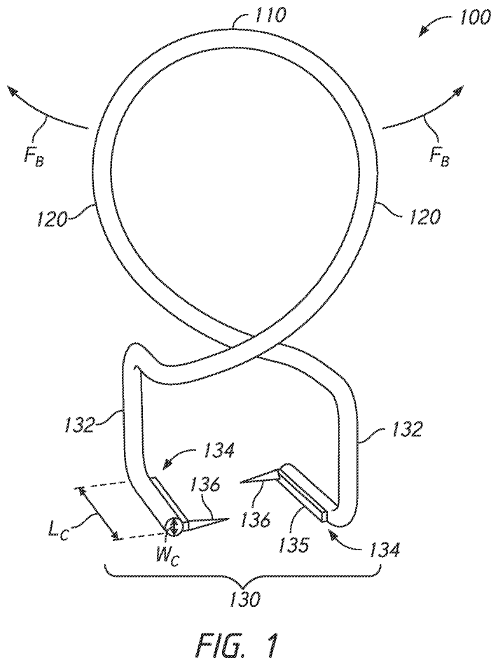

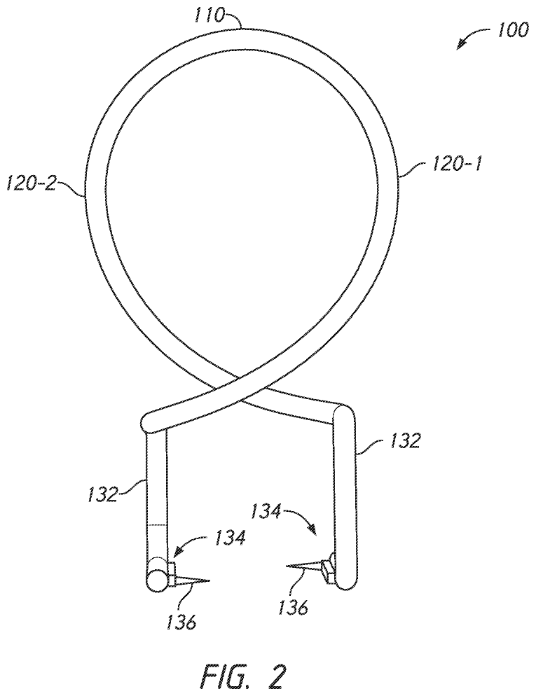

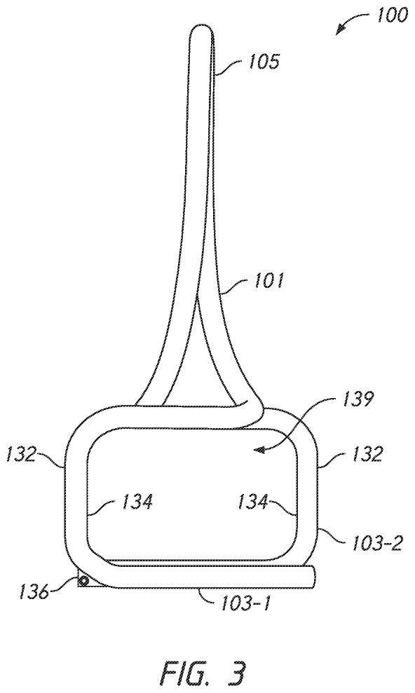

FIGS. 1-3 show an isometric, front, and side views, respectively, of an example suture clip according to the present disclosure. The suture clip 100 includes a spring element 110, two opposing side portions 120, and a clamp portion 130 that includes a pair of opposing closure elements 132 (i.e., first closure element 132-1 and second closure element 132-2). The spring element 110 exerts a biasing (or clamping) force FB, as shown by the arrows in FIG. 1, urging the spring element 110 towards its neutral (or unloaded) position and thereby urging the closure elements 132, which are operatively associated with the spring element 110, toward one another. The suture clip 100 is shown in FIGS. 1 and 2 in the open configuration. In the closed configuration, the generally circular loop defined by the spring element 110 and the side portions 132 would be slightly larger with the closure elements 132 positioned closer to one another, and in some cases with at least a portion of the closure elements 132 being in contact with one another.

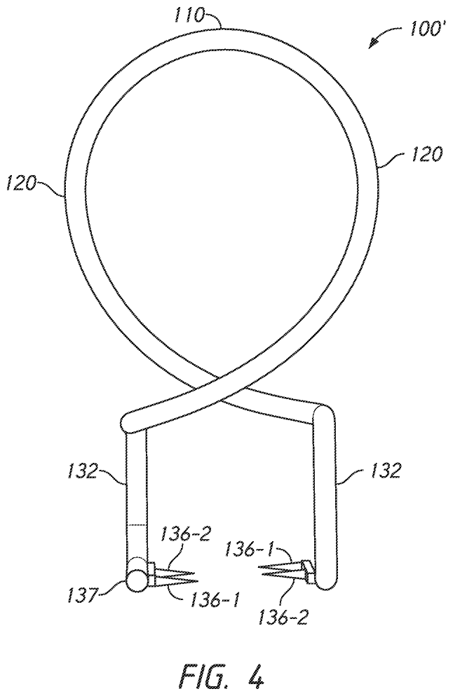

The suture clip 100 includes a pair of opposing closure elements 132. Each of the closure elements 132 includes a clamping surface 134 on the side of the closure element that faces the opposing closure element, and at least one spike 136 extending from the respective clamping surface 134. The clamping surface may extend along any suitable portion of, in some cases substantially along the entire, side of the closure element that faces the opposing closure element. The spike 136 may be operatively arranged with respect to the closure element such that it extends from the closure element toward the opposite closure elements. The spike 136 may be positioned anywhere where suitable along the clamping surface 134 and it may extend perpendicular to or at an angle to the clamping surface 134. In the example in FIG. 1, the spike 136 extends from the clamping surface 134, that is from the surface that contacts the tissue when the clip is in use, but in other examples, the spike may be attached to and extend from a different surface, such as the surface adjacent to the contacting surface and may be still be configured to suitably extend toward the opposing closure element. Furthermore, in the example in FIG. 1, each clamping surface 134 includes a single spike 136. However, in other examples, a greater number of spikes may be provided (e.g., two spikes per closure element as in the example in FIG. 4, or more), which may be arranged in any suitable desired pattern along the clamping surface.

In preferred embodiments, the clamping surfaces are long and narrow. The inventors have discovered that by making the clamping surface long and narrow, greater amount (e.g., a longer linear length) of tissue can be held together by the clip while still applying sufficient clamping force, thus promoting faster healing and reducing the risk of reopening of the incision. By having a relatively longer length along which clamping force is applied, the risk of fluid leakage is also reduced. By making the clamping surface relatively narrow (e.g., having a width which is significantly less than the length, such as at least 5 times, or in some cases 7 times or more, or 10 times or more than the length), the clamping force provided by the spring is more effectively transferred to the tissue, such as by concentrating the available spring force over a smaller total area. A clamping surface with this characteristic of being long and narrow (that is, having a relatively greater length than width) may also be referred to as being elongate in shape or having a high length to width aspect ratio (e.g., 5:1 or greater, 10:1 or greater, 12:1 or greater, 15:1 or greater). In some examples, the clamping surfaces can be continuous as shown or discontinuous, e.g., defined by a plurality of discrete contact regions (e.g., circular or differently shaped discrete regions) suitably arranged (e.g., in a line) over the same area as the continuous clamping surface in this illustrated example. In other words, the total clamping area may be defined by discrete contact regions which are arranged in a pattern that defines a high aspect ratio clamping area, although the individual contact regions themselves need not have high aspect ratio geometry. The total area over which clamping force is distributed may vary based on the specific application (e.g., the type of soft tissue being held together). Thus, the total area of each clamping surface may be tailored based on the specific surgical application for a given embodiment of the clip in order to provide sufficient pinch closure to avoid the incision opening due e.g., to fluid pressure. For example, a greater amount of force and thus a larger total clamping area may be needed for vascular tissue as compared to dural tissue. In the specific case of closing a dural incision, the fluid pressure may range from about 150 to 300 mm Hg, thus a suture clip designed for closing a dural incision may provide a clamping force of about 0.45N to about 0.8N, and in some examples a force of at least about 0.5N, or at least about 0.6N. The upper limit of the clamping force may be driven by the type of tissue being held. That is, parameters of the clip may be tailored (e.g., clamping force of the spring element and the configuration of the clamping surface) such that the force transferred to the tissue is less than a force that could cause trauma to the tissue. For example, the clamping surface and any texture provided thereon may be sufficiently blunt to facilitate clamping without cutting the tissue.

In the example in FIG. 1, the body of the suture clip 100 is made from a continuous piece of wire 101, such as titanium or NITINOL wire, having a suitable diameter to apply sufficient clamping force to the tissue, for example a clamping force of at least 0.5N. In other examples, different sizes of wires and forces may be appropriate. The wire 101 may be cut to length and bent to the desired shape (e.g., the serpentine shape shown in FIGS. 1-3). The opposite end portions 103-1, 103-2 of the wire 101 may be bent to a C-shape (as shown in FIG. 4), or an L-shape, to provide the opposing closure elements 132. The facing sides of the opposite end portions 103-1 and 103-2 of the wire 101, once shaped, may thus provide the clamping surfaces 134. The clamping surfaces may include any suitable portion of the C-shaped ends of the wire 101. In other embodiments, the closure elements may be configured to provide clamping surfaces defining a T-shaped or a Z-shaped contact area. The middle portion 105 of the wire 101 is looped into a generally circular shape to provide the spring element 110 and the side portions 120.

As previously described, the clamping surfaces may be long and narrow. That is, the surfaces that will contact the tissue may have a relatively greater length Lc than width Wc. The width Wc of the clamping surface in this example is the same as or smaller than the diameter of the wire 101, while the length Lc is defined by the lower leg of the C-shaped end portion of the wire 101. In this examples, the length Lc is about 2.5 mm. The length may be different in other examples, for example anywhere between 1 mm and 4 mm. In yet other examples, clips designed for joining different type of tissue may have different dimensions than those of the present examples. As discussed, the length and thus clamping area may be varied to suit different uses.

In the illustrated example, the clamping surface 134 includes a raised generally flat portion 135. The flat portion 135 may be formed by joining (e.g., laser welding) or forming (e.g., by additive manufacturing) additional material along the end portions of the wire. In other examples, a generally flattened portion may be formed by a cutting operation, such as laser or pressure-jet cutting the inner side of the closure elements to a flat profile. In yet other examples, at least a portion of the clamping surface 134 may be textured (e.g., by machining or molding the end portions into the desired texture) so as to increase the friction or gripping capability of the clamping surface 134. Regardless of the specific configuration of the clamping surface 134, the clip 100 additionally and preferably also includes at least one spike, which unlike texture added to the clamping surface has a significantly higher penetrative capability than the remaining clamping surface. In this manner, the spike(s) serves to more firmly secure the suture clip 100 to the tissue, such as by piercing the tissue and preventing lateral movement of the clamp with respect to the tissue. The spike(s), while illustrated as substantially perpendicular in the example in FIG. 1, may extend at any other suitable angle relative to the clamping surface.

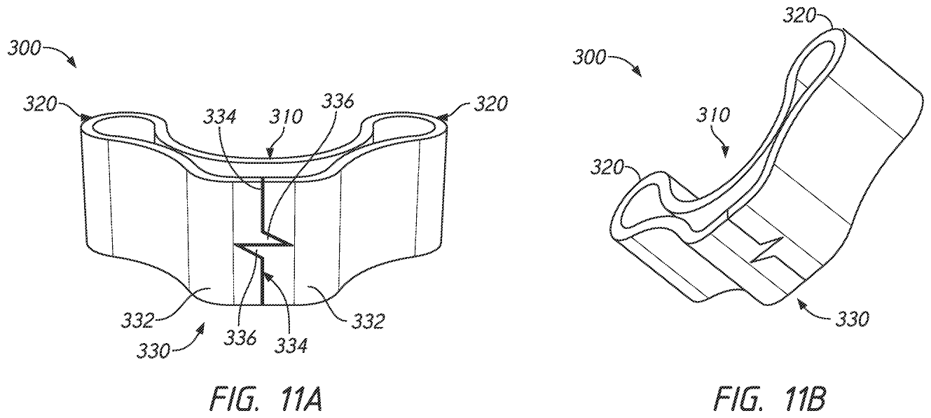

The clamping surfaces may have other suitable shapes. For example, the clamping surfaces, or portion(s) thereof, may be generally flat (e.g., as in the lobed suture clip in FIGS. 11A and 11B), and in some cases the clamping surface may be arranged so they are generally parallel to one another when clamping the tissue. In some cases, the surfaces may be generally flat but may be arranged to be angled relative to one another when clamping the tissue so as to exert a greater amount of force at one location (e.g., farther away from the incision end) than at a second location (e.g., closer to the incision end, or the reverse). In other examples, the clamping surface may be curved. For example, in some bent wire embodiments, the clamping surface may be provided by the rounded surface of the wire and thus the two opposing clamping surfaces may be arranged such that concave sides of the clamping surfaces face one another. In other examples, the curvature of the rounded surfaces may be reversed by forming a recess into the opposing clamping surface. Other geometries and relative arrangement for the clamping surfaces may also be used without departing from the scope of the present invention. The clamping surfaces 134 may have complimentary shapes, such that the two surfaces align (e.g., are substantially coextensive or overlie one another) when the clip is in the closed configuration, such that the forces applied by each of the opposing surfaces are generally aligned.

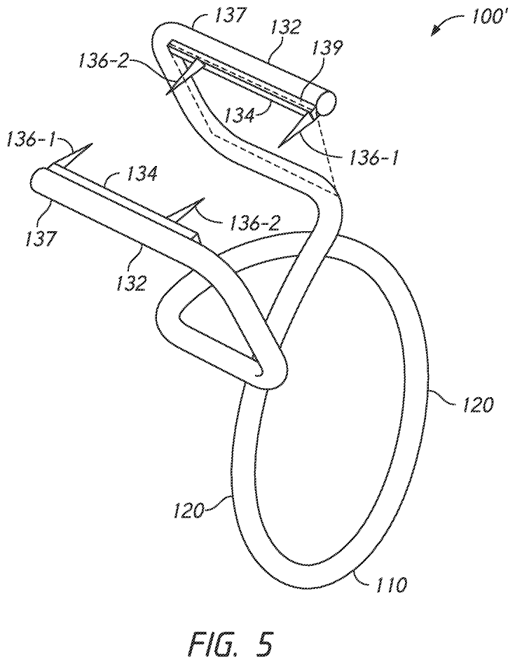

FIGS. 4 and 5 show another example of a suture clip 100′ according to the present disclosure. The suture clip 100′ is similar to the suture clip 100 described with reference to FIGS. 1-3. For example, the suture clip 100′ includes a spring element 110, two side portions 120, and two closure elements 132. In this example, the suture clip 100′ includes multiple spikes (e.g., 136-1 and 136-2) on each clamping surface 134. In this example, one of the spikes 136-1 is disposed at the end of the lower leg 137 of the closure element, and the second spike 136-2 is disposed along the length of the lower leg 137, spaced apart from the first spike 136-1. The spikes 136-1 and 136-2 are arranged along the opposing legs 137 such that they intermesh when the clip is in the closed configuration. Arranging the spikes so that the intermesh may enable a tighter closed configuration of the clip. In some examples, the spikes may be angled from the perpendicular direction, which may allow the clip to close even tighter with the spikes sliding against one another and/or the upper or lower surfaces of the lower leg for an even tighter closed configuration. In yet further examples, the clip may be configured to allow the clamping surfaces elements, or at least a portion thereof, to contact one another when the clip is closed (e.g., by providing apertures or grooves to accommodate the spikes therein when the clip is closed).

As illustrated in the examples in FIGS. 1-5, the first and second closure elements (or jaws) define first and second clamping footprints, respectively, with the first and second clamping footprints having complementary shape to one another. The clamping footprint (e.g., 139) can be understood to be the overall area circumscribed by the clamping surface of each closure element. As shown, the clamping surfaces need not extend or span the entire clamping footprint. In some embodiments, the clamping surface may extend only around a perimeter, or a portion of the perimeter, of the clamping footprint. The clamping footprints of each jaw may have complementary shapes, just as the clamping surfaces themselves. The clamping surface may be defined by multiple contact points arranged in any suitable pattern about (e.g., in a line around the perimeter) of the clamping footprint. In the examples in FIGS. 1-5, the closure elements 132 provide a generally rectangular clamping footprint 139, but in other examples the footprint may have a different shape such as a semi-circular or semi-ovular, or any suitable irregular shape. In some examples, depending on the total length of the clamping surfaces, two or more spikes may be provided for each millimeter of length.

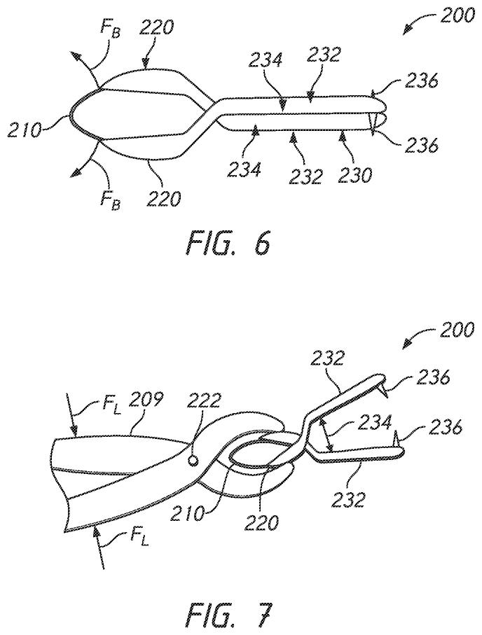

FIGS. 6-10 illustrate yet another example of a suture clip according to the present disclosure. Suture clip 200 includes a spring element 210, two opposing side portions 220, and a clamp portion 230, which includes a pair of opposing closure elements 232. The spring element 210 exerts a biasing (or clamping) force FB urging the spring element 210 towards its neutral (or unloaded) state thus urging the closure elements 232, which are operatively connected to the spring element, toward one another. The spring element 210 may be formed using a strip of metal, preferably a super-elastic alloy such as a shape memory alloy, which may be integrally formed with or joined to the side portions 220. In some example, the spring element 210 may be implanted using a leaf spring. In other examples, the spring element 210 may be a piece of wire (e.g., nickel titanium wire), which may be integrally formed with the rest of the clip or joined at each end to a respective one of the side portions 210. The suture clip according to the present disclosure may, in some embodiments, be formed form a unitary piece of material, such as a unitary piece of wire or strip of material (e.g., strip of shape memory alloy). In some examples, most of the suture clip (e.g., all components but the spike, as an example or other type of traction element(s)) may be made from a unitary piece of material and the remaining components (e.g., spike(s)) may be added thereto such as via any suitable joining technique or additive manufacturing technique.

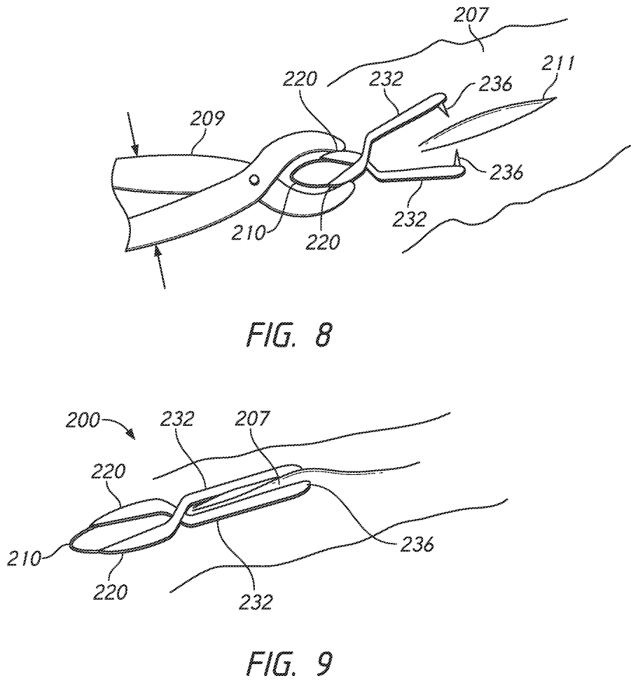

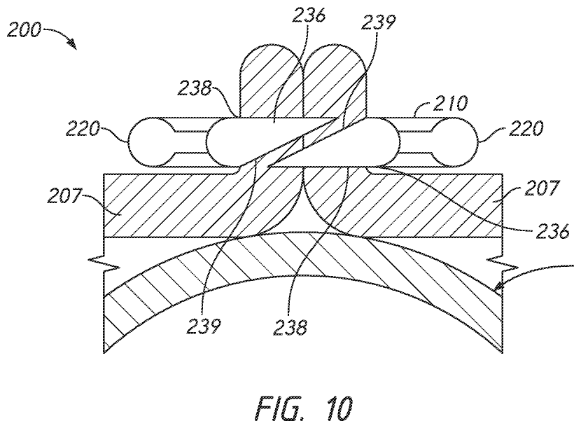

The suture clip 200 is shown, in FIG. 6, in a fully closed configuration and, in FIG. 7, in an open configuration (e.g., responsive to the application of a loading force FL applied by surgical pliers 209). Referring now also to FIGS. 8-10, while the clip 200 is shown fully closed in FIG. 6 (e.g., with the clamping surfaces 234 against one another), it will be understood that in use (see FIGS. 9 and 10), the clip 200 would be in a closed configuration in which the clamping surfaces 234 are not necessary in contact with one another but instead press against the tissue (e.g., tissue 207) placed between the clamping portion 230, thus clamping down on the tissue (e.g., holding the two portions of tissue 207 against one another). In some embodiments of clip 200, the ends of the two side portions 220 opposite the spring element may be joined at a pivot 222. Thus, when a loading force FL is applied to the clip 200, the side portions 220 are brought together causing the proximal end of the clip to deform into a more eccentric shape (e.g., from a circle to oval or from a relatively less eccentric to a more eccentric ellipse), thereby causing the closure elements 232 at the distal end of the clip 200 to spread apart, as shown in FIG. 7. During unloading of the clip, the clip 200 deforms in the reverse. That is, when the loading force FL is removed, the proximal end of the clip 200 defined by the spring element 210 and side portions 220 transitions to its neutral shape, which may be substantially circular or less eccentrically elliptical then when loaded, allowing the closure elements 232 to return to a closed position.

As shown in FIGS. 6-10, the suture clip 200 includes a pair of opposing closure elements 232. The closure elements 232 each include a clamping surface 234 on the facing sides thereof, and at least one spike 236 extending from the respective clamping surface 234. The spikes 236 may be positioned anywhere where suitable along the clamping surfaces 234. As with the previous examples, the clip 200 may be formed from a single piece of wire (e.g., NITINOL wire) formed into the desired shape, with a middle portion of the wire defining the spring and side portions, and the opposite ends of the wire defining the two closure element 232. In some examples, the portion of the wire providing the spring function may be flattened or otherwise shaped or modified to tailor the spring properties of the clip 200 as desired (e.g., to provide a sufficient amount of clamping force). In other examples, the spring element may be separately formed (e.g., from a strip of surgical-grade metal) and joined to the other components of the clip 200. Additionally, the spikes may be formed by bending and/or machining portions (e.g., the end portions) of the wire, or by a joining (e.g., laser welding) or additive manufacturing (e.g., laser sintering or other form of 3D printing) technique. Any suitable manufacturing technique may be used to obtain a suture clip of the shape and function described herein.

In use, the suture clips described herein (e.g., clip 100, 200) may be used to join tissue in a variety of surgical procedures. For example, the clip 200 (or any of the suture clips described herein) may be used to close a dural incision (e.g., following a craniotomy or spinal surgery), a vascular incision or other types of surgical incision. The clip 200 may be applied using conventional scissor-type pliers, which are used to squeeze the side portions 220 of the clip 200 together to open the clamp portion 230 (see e.g., FIG. 8). The surgeon then maneuvers the clip 200 into position relative to the incision 211 before releasing the force on the pliers. For example, the surgeon may align the clip 200 such that both sides of the clip are simultaneously positioned at a respective side of the incision, with each spike 236 being positioned sufficiently far into the tissue from the edge of the incision, such that upon release of the loading force, both sides of the clip simultaneously engage or clamp down on the tissue. Typically, the incision may be held temporarily closed (e.g., by clamps or forceps) while the clip 200 is maneuvered into position with respect to the incision and applied. In other examples, the surgeon may apply the clip by a hook and rotate technique, such as by hooking (e.g., by piercing one side of the incision with the spike(s)) one side of the clip and then rotating the clip over the incision to position the opposite side of the clip against the other side of the incision such that upon releasing of the spring, the spike(s) and clamping surface on the opposite side engages the other side of the incision, closing the incision. In this scenario, the incision may also be optionally temporarily held closed (e.g., by forceps) near the location where the clip is being applied, until the clamping force of the clip is applied to the tissue.

When the clip has been applied to close the incision, the clip may lie generally in line with the incision (e.g., as shown in FIG. 9) or may extend outward from the tissue 207 (e.g., out of the page in FIG. 9). In the example in FIG. 10, the spikes 236 of the suture clip 200 penetrate fully through the tissue 207 at the respective side of the incision and even partially into the tissue at the opposite side of the incision, although in other examples, the clip 200 may be configured to applied to tissue such that it does not fully penetrate the tissue 207 when closed. In either case, the spikes 236 advantageously function to prevent or minimize movement of the clip 200 relative to the tissue 207 or relative moment of the tissue at the opposite sides of the incision. The tissue at the incision can thus be held substantially immobilized by the clip promoting the self-healing process. Also as shown in FIG. 10, the spikes may be shaped for a cooperating fit with one another. In the example shown, each spike has a triangular profile with an outer surface 238 extending generally perpendicularly to the clamping surface and an inner surface 239 extending at an angle from the clamping surface and meeting the outer surface at an acute angle that defines the tip or point of the spike. The two inner surfaces of opposing spikes are so angled such that they are generally parallel relative to one another, each of the inner surfaces 239 providing a ramp for the inner surface of the opposing intermeshing spike 236, thus enabling a tight closure of the clip.

FIGS. 11-16 show further examples of a suture clip according to the present disclosure. The example suture clips in FIGS. 11-16 may, in some embodiments, be made from a unitary piece of material (e.g., a strip cut from a thin sheet of metal or a tubular section of tube stock material) subsequently shaped as shown.

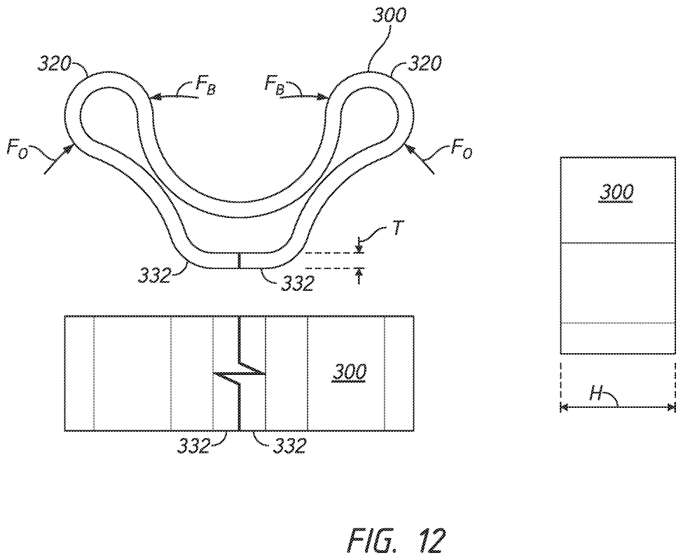

The suture clip 300 includes a spring element 310, two side portions 320, and a clamping portion 330. The spring element 310 exerts a biasing force, as shown by the arrows FB in FIG. 12, urging the clamp portion 330 toward the closed position (e.g., as shown in FIGS. 11A and 11B). In some embodiments, the entire clip, not just the spring element 310, exerts a biasing force to urge the clamp portion to the closed positions. For example, when using shape memory alloys, the entire shaped clip by virtue of the shape memory alloy's tendency to return to the memorized shape, will urge the clip to the closed position.

In the example in FIGS. 11A and 11B, the clamp portion 330 includes two opposing closure elements or jaws 332, each of which is joined to the spring element 310 via the respective side portion 320. Each of the jaws 332 includes a clamping surface 334 and at least one spike 336 extending from the respective clamping surface. The clamp portion 330 may be configured such that the spikes 336 intermesh allowing the clamping surfaces 334 to contact one another when the jaws are in the closed position.

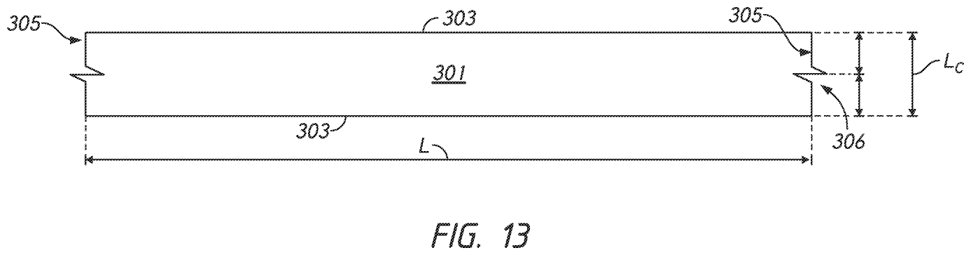

In this example, the suture clip 300 is formed from a strip of metal. The strip of metal may be cut from sheet metal stock in the pattern shown in FIG. 13 and bent to the desired shape shown in FIGS. 11-12. As with other examples herein, the suture clip 300 may be formed from a variety of surgical grade metals (e.g., titanium, nickel titanium alloy (NITINOL)) or from a polymer or composite material having suitable elastic properties (e.g., being capable of returning to a pre-loaded state upon release of a loading (or opening) force to apply a clamping force of at least 0.5N, preferably 0.6N or greater).

Finite element analysis was performed to develop suitable ranges for clip clamping force and to select suitable geometry for a suture clip according to the present disclosure. It was determined that in some examples, the material thickness of the clip may be at least 0.08 mm, at least 0.1 mm, at least 0.125 mm, at least 0.13 mm, or at least 0.15 mm. In some examples, the thickness of the material may be at most 0.15 mm, at most 0.18 mm, at most 0.2 mm, or at most 0.22 mm. As illustrated, in this example, the thickness of the strip of material may define the width of the clamping surface, while the height of the strip of material may define the length of the clamping surface. A diameter of the curved portion defining the spring element may be at least 0.8 mm, or at least 0.85 mm, or at least 0.9 mm, or 0.92 mm. The diameter may be at most 1.2 mm, at most 1.0 mm, or at most 0.95 mm. Also, the biasing force (or clamping force) applied by the spring element may be tuned to enable sufficient opening of the clip (i.e., to allow manipulation of the clip about the tissue and appropriate placement relative to the incision), while still providing sufficient clamping force to properly close the incision, in this example at least 0.5N of clamping force. In some embodiments, the clip may be configured to provide an effective opening (or separation of the two opposing closure elements) of at least 0.7 mm, in some cases, at least 0.75 mm. As discussed, the suture clip may be made from a variety of materials (e.g., surgical-grade stainless steel, titanium, titanium-nickel alloy or an alloy of cobalt-chromium-nickel-molybdenum (e.g., ELGILOY manufactured by Elgiloy Specialty Metals).

In some embodiments, preferably at least one spike or one pair of spikes of the clip is arranged relative to the clamping surface such that most (e.g., 85%, 90%, or more) of the clamping are is between the spike(s) and the edge of the incision. Thus, the spike serves to grab and secure the clamp with respect to the tissue relatively deeper into the tissue (i.e., away from the edge of the incision), while most of the clamping surface applies a clamping force along the region of tissue between the spike and the edge of the incision. In some examples, at least some of the clamping surface is on the side of the spike further away from the edge of the incision.

In one example, a suture clip for closing a dural incision (also referred to as dura clip) can be formed from a sheet of material having a thickness T from about 0.125 mm to about 0.145 mm. A strip 301 having an overall length L of about 9 mm may be cut (e.g., laser cut) from the sheet in the pattern shown in FIG. 13. The strip may have generally straight and parallel longitudinal sides 303 and opposite transverse sides 305, which are cut to include a lightning bolt (or zigzag) pattern 306. The portions of the transverse sides 303, which extend generally perpendicularly to the longitudinal sides 301 provide the clamping area of the clip 300, with the zigzag pattern defining the spikes 336. The zigzag pattern is cut at each of the opposite ends of the strip 301 such that the spikes 336 intermesh when the strip 301 is formed into the lobed suture clip 300. That is, the spikes 336 are sized and positioned along the length of the transverse sides 303 such that the spikes 336 fit with one another allowing the clamping surfaces 334 to contact when the clip 300 is the closed configuration.

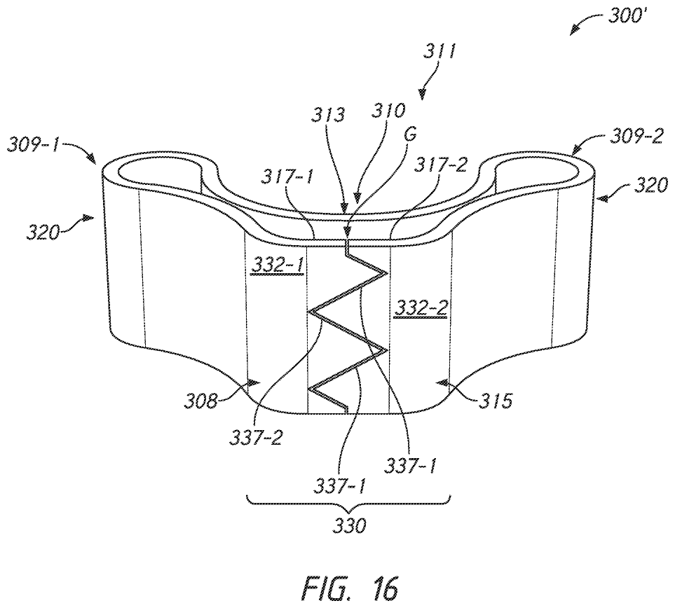

To form the strip 301 into the lobed shape of clip 300, the strip 301 may be provided into a mold which has a negative shape to the lobed shape of clip 300. While being formed into the desired shape, the strip 301 may be heated so that the shape memory material may be “imprinted” with the lobed shape as the new neutral or nominal shape of the material to which the material would return whenever unloaded. Alternatively, the clip 300 may be formed from tubular stock material with a thickness T about 0.12 mm to about 0.15 mm. A cylindrical section is cut from the tubular stock material. The cylindrical section may have any suitable height H selected to provide the desired elastic properties of the clip 300, in this case a height of about 1 mm. In other examples, different thickness, height, or circumferential length of the source material may be used. As will be understood, the parameters of the stock material and/or resulting clip (e.g., material thickness, circumferential length, height, and relative dimensions of the lobes and curvatures of the lobes) may be tailored to configure a suture clip with clamping force suitable for any particular application as may be desired. Returning back to the current example, once the cylindrical section has been cut to the desired length, the cylinder may be formed by any suitable forming technique, such as by pressing, shaping, bending, molding, or any combination thereof. Once the cylindrical section has been formed into the lobe shape shown e.g., in FIGS. 11A and 11B, a cut in the lightning bolt pattern (or other closure pattern, see e.g., FIG. 16) may be formed through the thickness of the material to create the clamping portion 330.

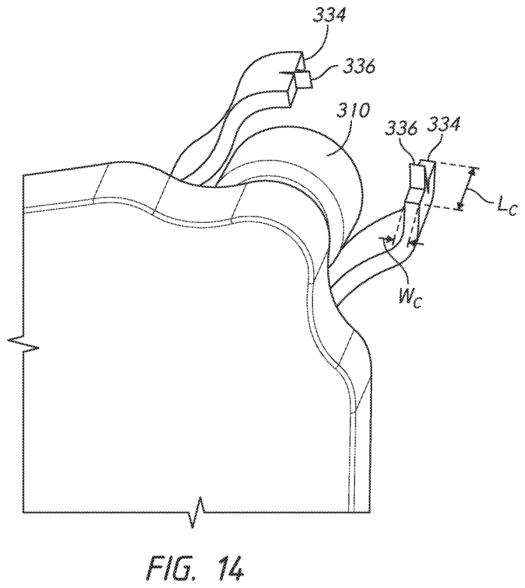

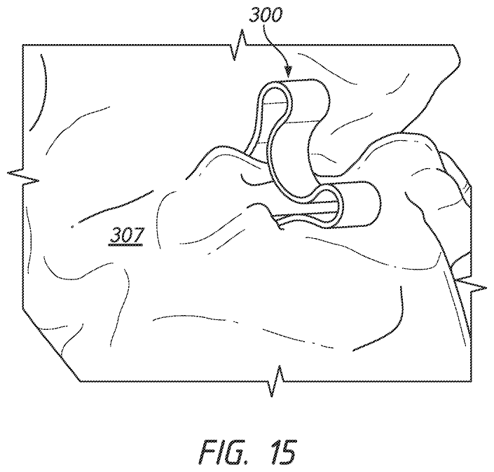

FIG. 14 shows the suture clip 300 in an open configuration with the clamping surfaces exposed, and FIG. 15 shows the suture clip 300 applied to dural tissue. FIG. 16 shows a suture clip 300′ which similar to the suture clip 300 and may include most or all of the components of suture clip 300, such as a spring element 310, two side portions 320, and a clamp portion 330. In this example, the clamp portion 330 of the suture clip 300′ includes non-piercing traction elements 337 arranged with respect to (e.g., projection in relation to) the clamping surface. The non-piercing traction elements 337 are defined by peaks 337-1 and valleys 337-2, which define a toothed-type closure at the interface between the two closure elements 332-1 and 332-2.

As described, a suture clip according to the present disclosure (e.g., clip 300, 300′) may include a clip body that is made from a strip of material (e.g., metal or resiliently elastic composite), with the strip of material being formed into a closed-loop shape having first and second lobe ends or portions (e.g., 309-1, 309-2) spaced from one another, and a middle portion 311 connecting the first and second lobe portions. The middle portion may include a spring side 313 and clamp side 315, with the spring side 311 being configured to apply a biasing force to urge the first and second lobe ends away from one another. The clamp side may define a gap G between opposite ends 317-1, 317-2 of the strip of material such that the opposite ends can be spaced apart responsive to application of a loading (or opening) force against the biasing force to allow the clip to be provided in an open configuration. The clamp side 315 includes at least one spike (e.g., spikes 336-1 through 336-4) at the gap configured to at least partially penetrate soft tissue positioned between the opposite ends 317-1 and 317-2 of the shaped strip. The clamping area provided by the two jaws 332-1 and 332-2 may be tailored as appropriate for a given surgical application. As illustrated, the opposite ends of the strip may have complimentary shapes such that they intermesh with one another when the strip is formed into the closed loop shape of the lobe-shaped design. In some examples, the opposite ends may be shaped in a complimentary zig-zag pattern defining at least one pair of opposing spikes.

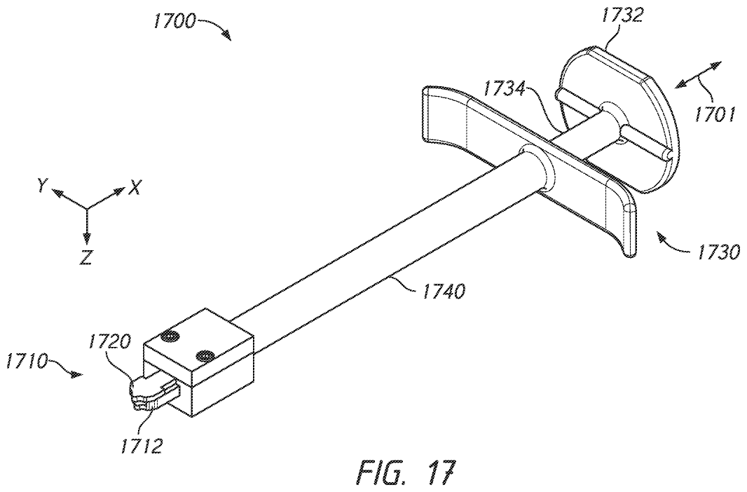

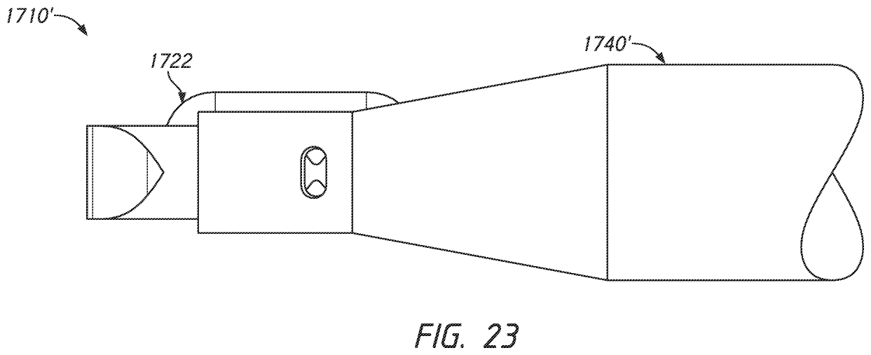

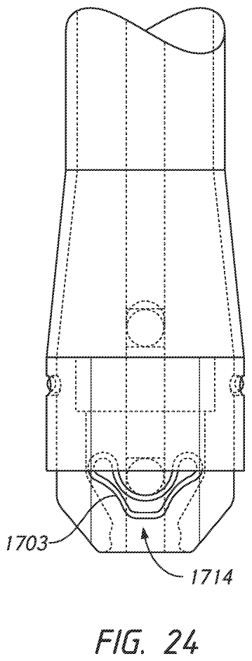

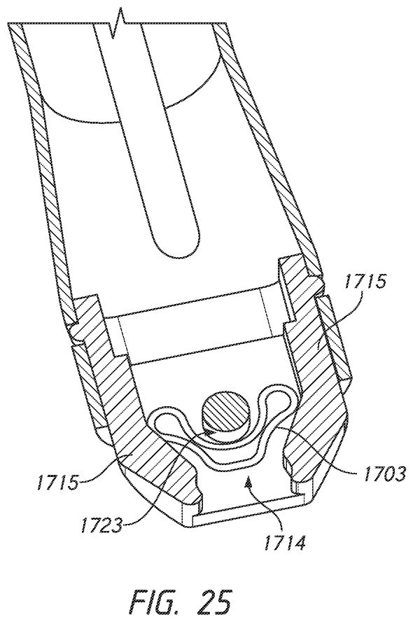

As can be further seen, the suture clips 300, 300′ have a generally v-shaped body, with the spring element and the clamp portion defining the apex of the v-shaped body, and the two side portions (at the lobes) defining the two angled legs of the v-shaped body. To load (or open) the suture clip, the two angled legs are deformed toward one another, and conversely, during unloading of the clip (allowing it to close under the biasing force of the spring element), the two legs return to their neutral (or starting) position. The loading and unloading of the clip may be performed by hand or preferably by a tool, such as a suture clip applier (e.g., as in any of the examples described further below with reference to FIGS. 17-26). In other examples, conventional surgical pliers or forceps can be used to open the clip and hold it open while the surgeon manipulates it into plate in relation to the tissue.

In accordance with further examples of the present disclosure, a tool for applying a suture clip (e.g., clip 300) may include a working tip at a distal end of the tool and a handle at a proximal end of the tool. As is conventional when referring to relative positioning on a surgical instrument, the term “proximal” refers to the end of the apparatus which is closer to the user and the term “distal” refers to the end of the apparatus which is further away from the user.

In some embodiments, the working tip includes a clip receiving channel configured to accommodate the clip at least partially therein. The clip receiving channel may be shaped such that it is wider at the proximal end than at the distal end. As such, the clip receiving channel may be shaped such that a proximal end of the channel is able accommodate the clip in an unloaded (or closed) configuration, while a distal end of the channel is only able to accommodate the clip when loaded (i.e. opened). In some such embodiments, the working tip also includes a pusher movable relative to the clip receiving channel. As will be further described, the clip receiving channel and the pusher may both be supported on a frame of the tool where at least one of the two is movably coupled to the frame such that the pusher and the clip receiving channel are movable in relation to one another. In some embodiments, the pusher may be movably coupled to the frame, while in other embodiments the same effect may be achieved by the pusher being stationary to the frame while the clip receiving chamber is defined in a component that is movable to the frame. As will also be further described, the handle of the tool is operatively connected to the working tip such that operation of the handle causes one of the clip receiving channel and the pusher component to move relative to the other one of the clip receiving channel and the pusher to advance the clip along the clip receiving channel while simultaneously opening a clamp portion of the clip.

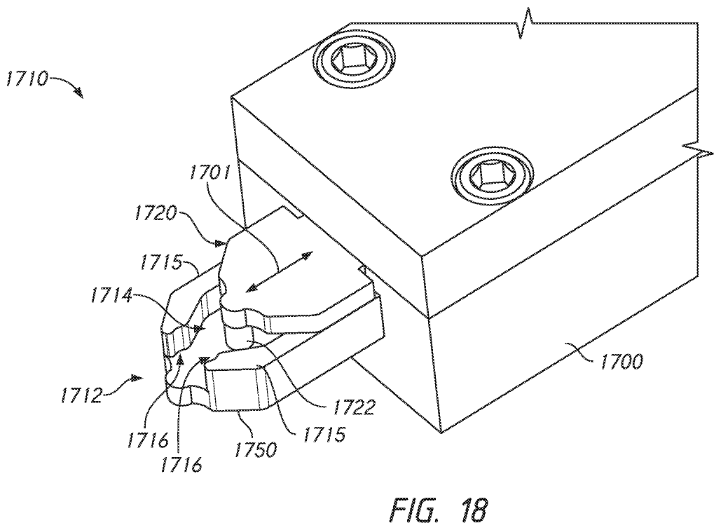

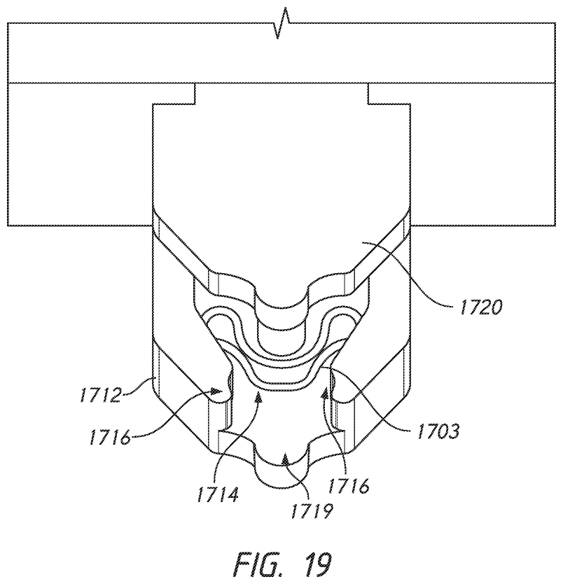

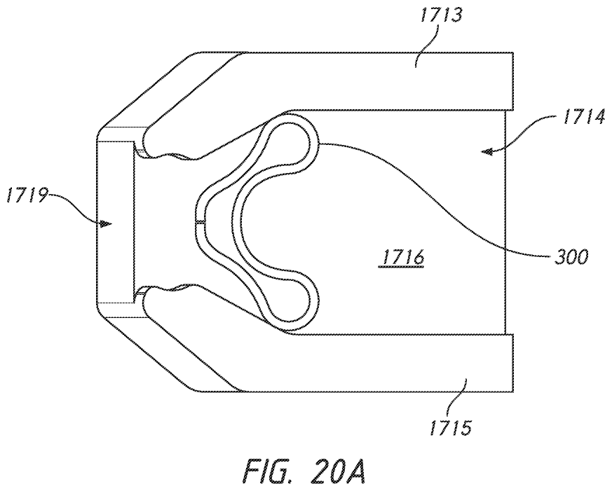

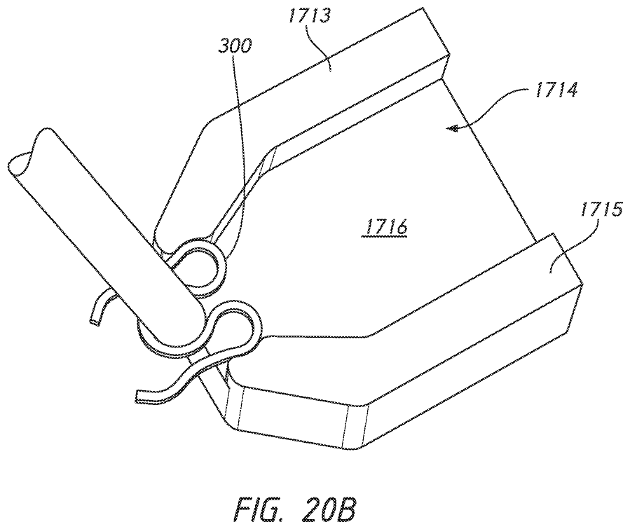

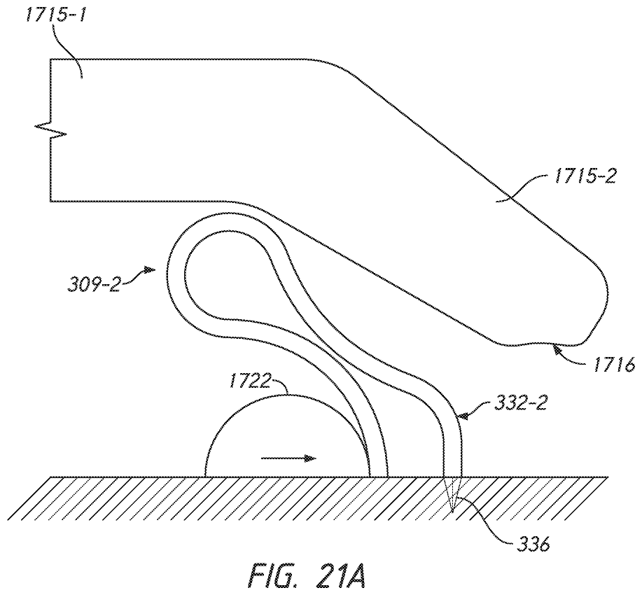

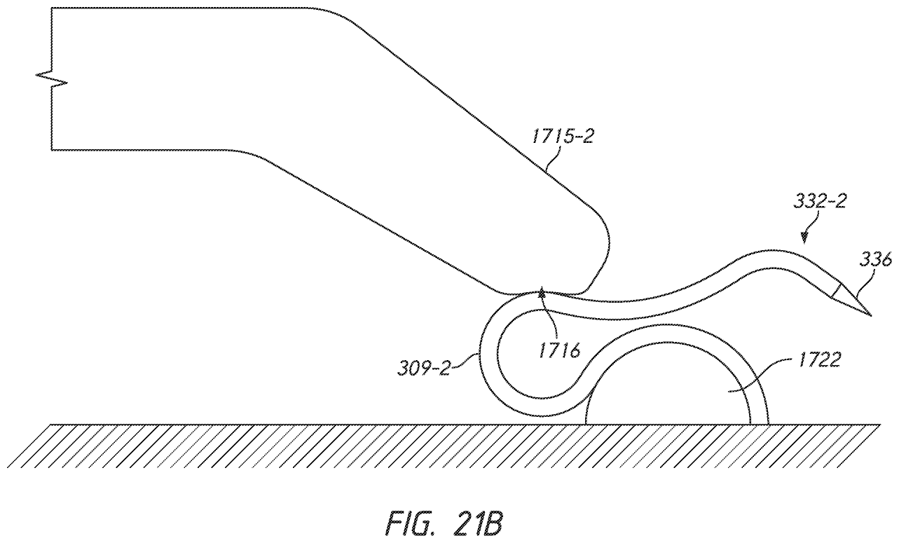

FIGS. 17-21 show views of a tool for applying a suture clip in accordance with some examples of the present disclosure. Specifically, FIG. 17 shows an isometric view of a suture clip applier 1700. FIGS. 18 and 19 show partial isometric and front isometric views of the working tip 1710 of the applier 1700 of FIG. 17, with an outline 1703 of an example clip disposed in a starting position (before loading) within the tool. FIGS. 20A and 20B show an enlarged top down view of a suture clip in a prototype clip receiving chamber in accordance with some examples herein, and FIGS. 21A and 21B shows diagrammatic views of a portion of the working tip 1710 illustrating the operation of loading (or opening) the clip (in FIG. 21B) from it unloaded (or closed) configuration (in FIG. 21A).

As shown in FIGS. 17-21, the suture clip applier 1700 includes a working tip 1710 specifically configured to apply a lobed suture clip according to the present disclosure, for example the suture clip 300 or 300′ show in FIG. 11A and 16, respectively. The working tip 1710 includes a clip receiving component 1712 and a pusher component 1720, which are together operable to load (or open) the suture clip before it can be applied to animal tissue (e.g., Dural tissue 307). The working tip 1710 is operatively connected to a handle 1730, which in this example is implemented as a plunger-type handle including a handle end 1732 connected to a plunger 1734. In other examples, the clip receiving component and/or pusher component of the working tip may be differently actuated, such as using a scissor-type handle or any other suitable manipulation device configured to move one of the clip receiving component and pusher component relative to the other.

The plunger 1734 may be at least partially received within a housing 1740. The housing may include a cylindrical (or substantially cylindrical) portion, which receives the plunger 1734 through a passage. The housing may include a distal enclosure that may include a biasing component configured to bias the plunger 1734 toward the handle end of the housing. The applier may be used for the application of a suture clip during open procedures or during an endoscopic procedure. During an endoscopic procedure, surgery is performed in a hollow viscus of the body through an endoscopic tube inserted through a penetration through the skin. The penetration is typically made with a trocar and a cannula is inserted therethrough providing a port for the insertion of surgical instruments, and in this case for also inserting a portion of the suture clip applier. Thus in some examples, the applier may be sized and shaped such that at least a distal portion of it fits through a surgical port provided, e.g., by a trocar.

The clip receiving component 1712 includes a clip receiving channel 1714 configured to accommodate the suture clip (e.g., clip 300) at least partially therein. In the illustrated example, the clip receiving channel 1714 is an open channel defined by a base 1716 of the clip receiving component 1712 and two side walls 1713 and 1715 arranged opposite one another and spaced apart from one another. Each of the side walls includes a proximal section 1713-1, 1715-1 and a distal section 1713-2, 1715-2 which is at an angle to the proximal section. As shown, the side walls converge distally to push against the lobes of the clip as the clip is slid forward in the channel. As such the proximal section of each wall provides a ramp for the sides of the clip as the clip is advanced along the channel 1714. The proximal sections are angled toward one another such that the clip receiving channel 1714 defined between the walls has a proximal portion that is wider that its distal portion. The wider proximal portions is sized to accommodate the clip (e.g., clip 300) in an unloaded state, while the narrower distal portion is sized to only accommodate the clip when loaded (e.g., opened).

The pusher component 1720 includes a pusher 1722 operatively connected, in this case fixed via the plunger 1734, to the handle end 1732, such that the application of force (e.g., by the surgeon) to the handle end 1732 displacing the handle end along the longitudinal direction 1701 causes the pusher 1722 to also displace along the longitudinal direction 1701. The pusher 1722 in this example includes a post which is sized to engage a rear wall the clip, e.g., to stabilize the clip as the clip is advanced and opened by the applier 1700. Additionally, the side walls 1713 and 1715 include detents 1716 to “catch” and hold the clip (e.g., clip 300) in the opened configuration while the surgeon is applying it to the tissue before the clip is released and allowed to return to the closed configuration to clamp down on the tissue.

In this example the clip receiving component 1712 remains stationary relative to a frame of reference of the applier 1700, while the pusher component 1720 is actuated (e.g., translated back and forth along direction 1701). In other examples, different arrangements may be used which effect the same relative movement between the channel 1714 and the pusher 1722. For example, the pusher component 1720 may be the component that remains stationary, while the clip receiving component moves. In other examples, both components 1712 and 1720 can move relative to one another and the stationary frame. That is, in some examples, the portion of the applier that includes the clip receiving channel 1714 may be actuated in relation to the portion that includes the pushed 1722, which in this example may remain fixed relative to the reference frame (e.g., XYZ reference frame in FIG. 17) of the applier 1700. Other suitable configurations may be used for the handle 1730, for example a scissor-type handle, whereby the opening and closing of the handle may be translated, e.g., via one or more linkages and/or pivotal joints, to a translating motion of the pusher 1722.

As shown in FIGS. 18-20, the proximal end of the channel is wider than the distal end. The proximal end is sized to accommodate the clip in an unloaded state, while the distal end is sized to accommodate the clip only in a loaded (open) state. The narrowing of the channel is achieved by angling the proximal wall portions of the two side walls toward one another (i.e., toward the centerline of the channel). In this manner, the side walls essentially define a ramp for each side of the clip as the clip is advanced along the length of the channel toward the distal opening 1719.

Additionally, the clip receiving component is provided with detent feature(s) 1716 configured to at least temporarily retain the clip in the open configuration. In the illustrated example, the detent features are provided in the side walls. Specifically, each of the side walls has a rounded distal end and the detent 1716 or catch is in the rounded portion of the side wall facing the opposite side wall. In use, as the clip is advanced along the channel 1714, the side portions of the clip are brought toward one another by the advancing of the side portions of clip into the narrowing part of the channel (i.e., up the ramps) until each side portion engages the respective detent 1716. The detent 1716 is implement as a recess in the otherwise outwardly (or concavely) rounded portion of the wall. The recess is shaped for a cooperating fit with the sides of the clip. That is, the curvature of the recess matches the curvature of the side portion such that as the side portions advance along the ramps, the side portions will each engage its respective detent and will be held into the detent 1716, by virtue of the spring force of the clip causing the side portions to spread ever so slightly when encountering the detent and to push outward toward the side walls in the detent, until the clip is further advanced by further application by the loading component of sufficient forward force to advance beyond the bump at the leading of the detent.

In some embodiments, a cartridge 1750 that holds a stack of suture clips may be removably coupled to the applier 1700 and a clip feeder mechanism, in the applier or in the cartridge may feed individual clips from the cartridge into the chamber. In some examples, the cartridge may be attachable proximate to the working tip and the clips may be fed through an opening in the base of the channel 1714. In other examples, the cartridge may be attachable to the body of the applier, such as between the working tip and the handle and the clips may be fed through the proximal end of the channel. In yet further examples, the cartridge may be integrated with the tool. That is, a clip loading chamber may be built into the applier, such as in the housing 1740, and stacked clip refills may be loaded into the built-in clip loading chamber. In yet other examples, particularly with scissor or plier-type appliers, individual clips may be manipulated one at a time before another clip is picket up and manipulated by the applier.

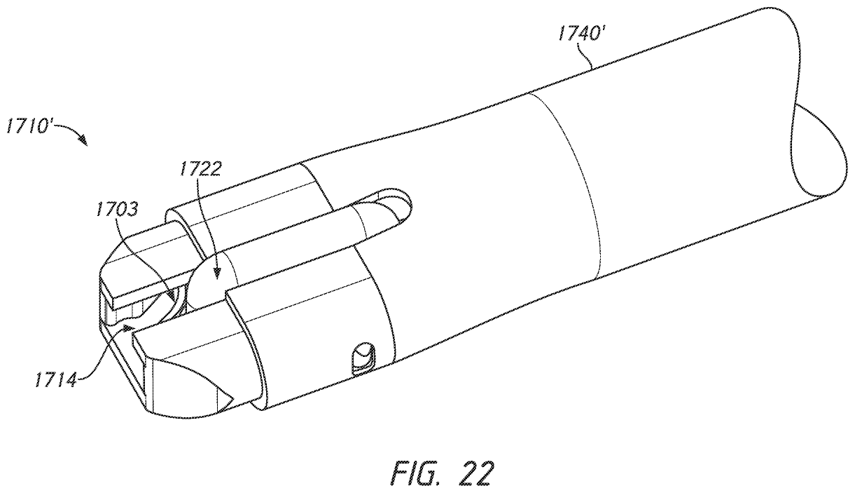

FIGS. 22-25 shows views of the working tip 1710′ of an applier according to a further example of the present disclosure. The applier in this embodiment has a similar configuration to applier 1700 in that it includes a clip receiving chamber 1714 and a pusher 1722, which are movably coupled to one another and operatively connected to the housing 1740′, which also extends proximally towards the handle (not shown). Either the clip receiving chamber 1714 or the pusher 1722 may be movable relative to the housing 1740′, which defines a stationary frame of the applier. The applier may be plunger-style applier similar to the one in FIG. 17, or it may be a scissor-type applier. In the case of a scissor-type applier, the moving component at the working tip 1710′ may be operatively connected to a movable part of a scissor-type handle or to a trigger of the handle such that a squeezing action of the trigger or scissor handle causes relative movement between the clip receiving chamber 1714 and pusher 1722. Similar to the applier 1700, the pusher 1722 includes a clip engagement end 1723 that is shaped for a cooperating fit with the rear side (i.e., opposite the clamp side) of the clip. The clip engagement end 1723 may be sized and shaped to also stabilize the clip as the clip is advanced along the channel 1714

The clip engagement end 1723 and the spring element may have suitably sized curvatures to allow the clip engagement end 1723 to fit into the curved portion defined by the spring element of the clip 300 to stabilize the clip as it is being advanced. The curvature at the clip engagement end 1723 may be smaller than that of the spring to allow the spring's curvature to reduce as the spring wraps around the clip engagement end 1723 upon advancement, as shown in FIG. 20B), but the curvature of clip engagement end 1723 may be sufficiently large to enable the pusher 1722 to apply the force needed to advance the clip and to also stabilize the clip during advancement. Thus, the pusher 1722 in this example serves a dual purpose of advancing the clip along the channel (i.e. by pushing the clip down the length of the channel) while simultaneously stabilizing the clip as the two side portions are squeezed by the walls to open the clip. In the open configuration in FIG. 20B, the clip 300 is in a stable open configuration. As shown in FIG. 20B, once the clip has advanced sufficiently along the channel such that the rear side of the spring (also referred to as post engagement area) has wrapped more than 50% around the post, the clip is provided in a stable open position by virtue of the lobes wrapping around and holding on to the back of the post. The clip can stably remain in this position (without closing as the surgeon maneuvers the clip into place) until the pusher is advance further to advance the clip over the bumps of the detents. The applier 1700 is suitably designed so that the location of the detents is appropriately matched with the location of advancement of the post where this wrapping about occurs so that the clip can be stably held open by both the detents and the wrapping of the lobes behind the post.

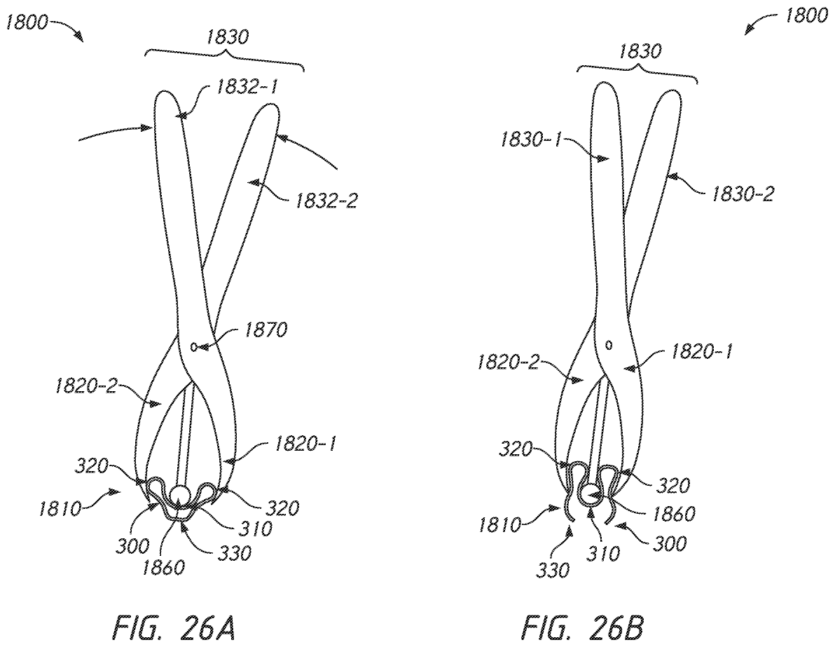

FIGS. 26A and 26B show a suture clip applier in accordance with further examples of the present disclosure. The suture clip applier 1800 is implemented as a plier-type tool with a handle 1830 that includes two pivotally connected handle ends 1832-1, 1832-2 and a working tip 1810 with two pivotally connected holding portions 1820-1, 1820-2. The suture clip applier 1800 is adapted to manipulate a suture clip (e.g., clip 300) which has a curved spring element 310 having its concave side oriented toward the clamp portion 330 of the clip. The applier 1800 include a working tip 1810 at its distal end. The working tip 1810 includes opposing holding portions 1820-1, 1820-2 configured to engage respective ones of the opposing actuation sides 320 of the clip such as to hold the clip 300 between the holding portions and for loading the spring element 310 and thus opening the clip. The applier 1800 also includes a stabilizer 1860 positioned between the two holding portions 1820-1, 1820-2. The stabilizer can be implemented as any suitable type of structure that includes a distal end shaped for a cooperating fit with the convex side of the spring element 310. For example, the stabilizer may be a rod with a rounded rod end that has a suitable curvature to fit in and allow the spring element to wrap around it during opening of the clip. As such, the stabilizer 1860 contacts the spring element 310 when the clip is held at the working tip 1810. As the two sides of the clip 300 are squeezed together, the rear side of the spring element wraps around the stabilizer 1860, as shown in FIG. 26B. That is, the curvature of the spring element 310 reduces to match, at least along a portion of the spring element, the curvature of the distal end of the stabilizer 1860. In some embodiments, the stabilizer 1860 is operatively coupled to the body of the applier 1800 (e.g., via a rack and pinion gear engaged with the pivot 1870 or some other type of mechanism) such that the stabilizer 1860 moves forward (away from the pivot) when the handle ends 1832-1, 1832-2 are squeezed. As such the stabilizer 1860 can remain in contact with the spring element 310, continuing to stabilize the clip while the clip is being opened.

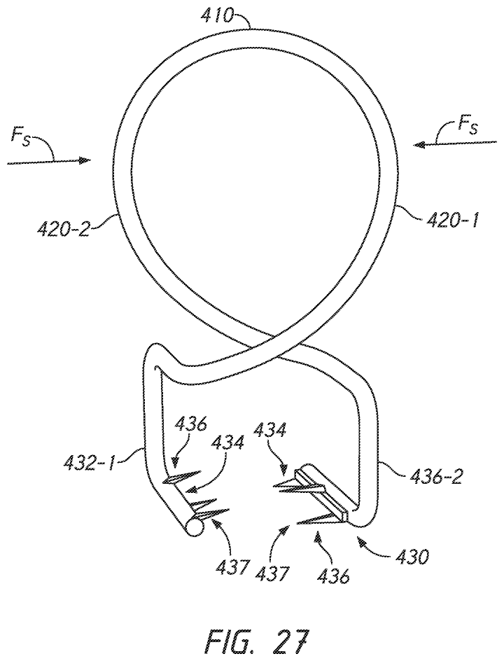

FIGS. 27-29 show another example of a suture clip similar to the examples describe with reference to FIGS. 1-5. The suture clip 400 includes a manipulation portion 420 configured for actuation by the user to apply a squeezing force Fs as shown the arrows to open the clamp portion 430. The first and second ends of the spring element 410 are joint to the respective one of the first and second actuation elements 420-1 and 420-3, respectively. The clamp portion 430 includes a first and second closure elements 432-1 and 432-2 (also referred to as jaws 432-1 and 432-2), which are configured to hold or secure the two portions of the tissue (e.g., the two sides of the incision) tightly together to prevent movement or separation of the tissue. The jaws 432-1 and 432-2, which are biased, by the spring element, towards one another such that they are substantially against one another (e.g., as shown in the cross-sectional view of FIG. 29) in the closed position, are configured to firmly hold the tissue therebetween through the application of inward pressure (i.e., toward one another). Like the suture clip of the earlier examples, the suture clip 400 may be formed from a unitary length of wire (e.g., NITINOL or other shape-memory material wire) which is shaped into the configuration shown in FIG. 27.

Suture clips according to the present disclosure may have any suitable number of traction elements (e.g., spikes) on each of the clamping surface. For example, as shown in FIG. 27, at least three traction elements 437 may project with respect to each of the clamping surfaces 434. The traction elements 437 in this example are implemented as spikes 436. In other examples, the traction elements 437 may be implemented using any other suitable structures such as hooks, substantially cylindrical prongs or tines, or other types of protruding structures, which may be pointed, blunt, and/or textured. The traction elements may be provided around the perimeter of the respective clamping surface and arranged in any suitable pattern such that they do not overlap with the traction elements on the opposite clamping surface. In the example in FIG. 27, one of the clamping surfaces is provided with two spikes along the proximal edge and one spike along the distal edge, while the opposing clamping surface is provided with a single spike along the proximal edge and two spikes along the distal edge of the clamping surface. The misalignment of the individual traction elements allows the clamp portion to close more tightly, e.g., as shown in the cross sectional view in FIG. 29, thus enabling the suture clip 400 to hold the tissue more firmly, with the spikes further serving to prevent relative movement between slippery bodily tissue and the suture clip.

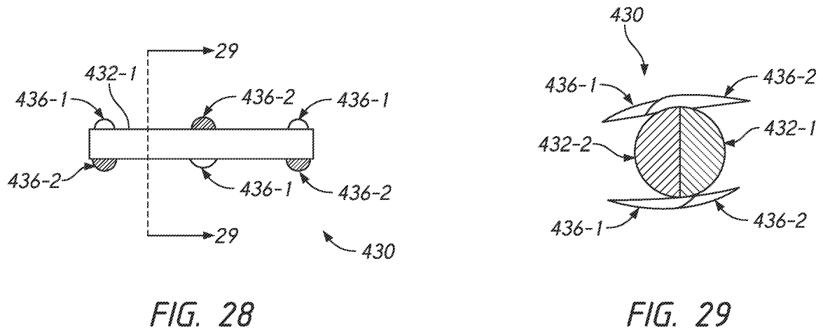

The suture clip 400 is shown in FIG. 27 in the open position, the suture clip being configured to spring back to the closed position when the squeezing force (Fs) is removed. In this example, the individual spike are arranged along the proximal and distal edges of the clamping surfaces such that when the clip 400 is in the closed position, the spikes of the opposing jaw extend over the respective proximal and distal edges of the clamping surfaces 434. This can be perceived more clearly from FIGS. 28 and 29, which show partial side and cross-sectional views of the clamping portion 430. FIG. 28 show the clamping portion viewed from the side of the first jaw 432-1. The first jaw 432-1 includes the spikes 436-1, two of which are positioned along the proximal edge and a third one positioned along the distal edge at a location between the two upper spikes. The second jaw, not visible in this view, includes the spikes 436-2, two of which are positioned along the distal edge and the third one positioned along the proximal edge of the second clamping surface, in a similar but inverted pattern to that of the first jaw 432-1. The spikes 436-1 of the first jaw 432-1 extend from the first jaw toward the second jaw (i.e. into the page) passing over the top and bottom sides of the second jaw, while the spikes 436-2 of the second jaw (not visible in this view) extend from the second jaw toward the first jaw (i.e. out of the page) passing over the top and bottom sides of the first jaw 432-1. Referring also to the cross-sectional view in FIG. 29, the vertical distance between the spikes 436-1 of the first jaw is just large enough to allow the spikes 436-1 to clear the edges of the second jaw and the vertical spacing between the spikes 436-2 is similarly just large enough to allow the spikes 436-2 to clear the edges of the first jaw, thus allowing the jaws to close in a tightly fitting configuration as shown in FIG. 29. While the jaws of the clamping portion are show to have a semi-circular cross section in this example, the jaws may have different cross-sectional shapes such as semi-ovular, rectangular, triangular, or other suitable shapes. In some examples, each of the clamping portions may be formed by flatting an end portion of the single piece wire, before or after the wire has been shaped, and the traction elements may be joined to or formed onto the wire by any suitable technique such as by fusing, laser welding, or via additive manufacturing.

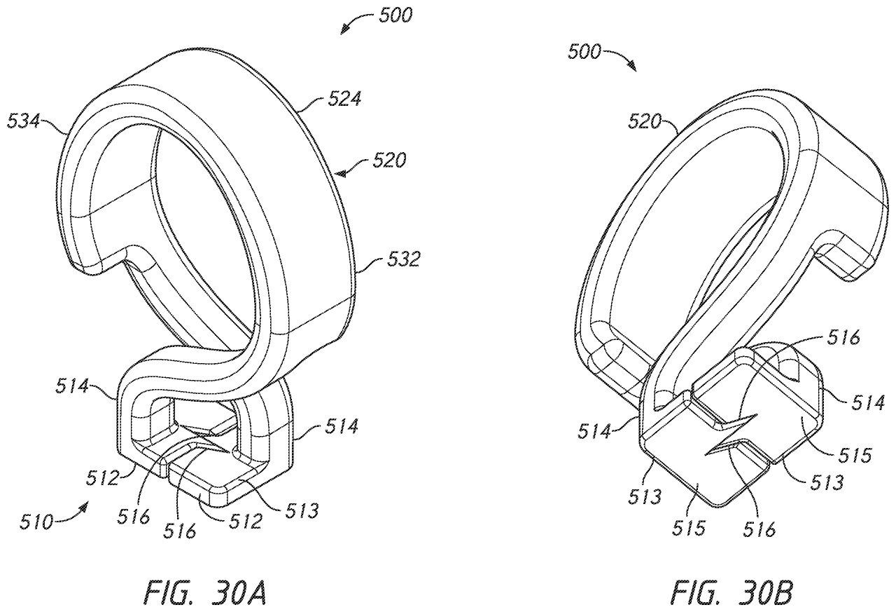

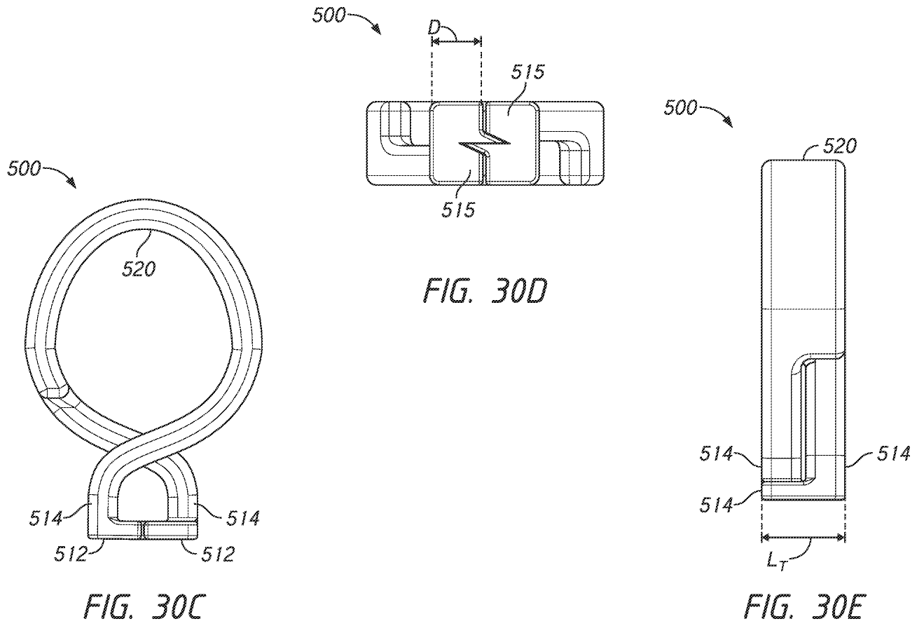

FIGS. 30-36 disclose further embodiments of suture clips in accordance with the principles of the present disclosure. Each of the suture clips in these embodiments includes a clamp, which is configured for clamping two portions of biological tissue, such as two sides of a Dural incision for closing and aiding in the healing of the incision. The clamp of the suture clip is held in the clamped (or closed) position by a spring force. The spring force may be applied by a spring (e.g., spring 520 of suture clip 500, or spring 920 of suture clip 900 described further below). The spring force of the spring may be augmented by the tendency of a shape memory material to return to a memorized shape, e.g., in examples in which the suture clip is formed, at least partially, from a shape memory material (e.g., a shape memory alloy such as nickel-titanium alloy or another suitable shape memory alloy) and the closing force exerted by the clamp may, at least in part, result from the memorized shape of the material. Shape memory materials “remember” a shape and thus tend to return to that memorized shape following elastic deformation. This memory effect may function alone to apply a closing force or may augment a natural spring force that may be embodied into the suture clip by the shaping of the clip (e.g., by embodying a spring element into the clip).

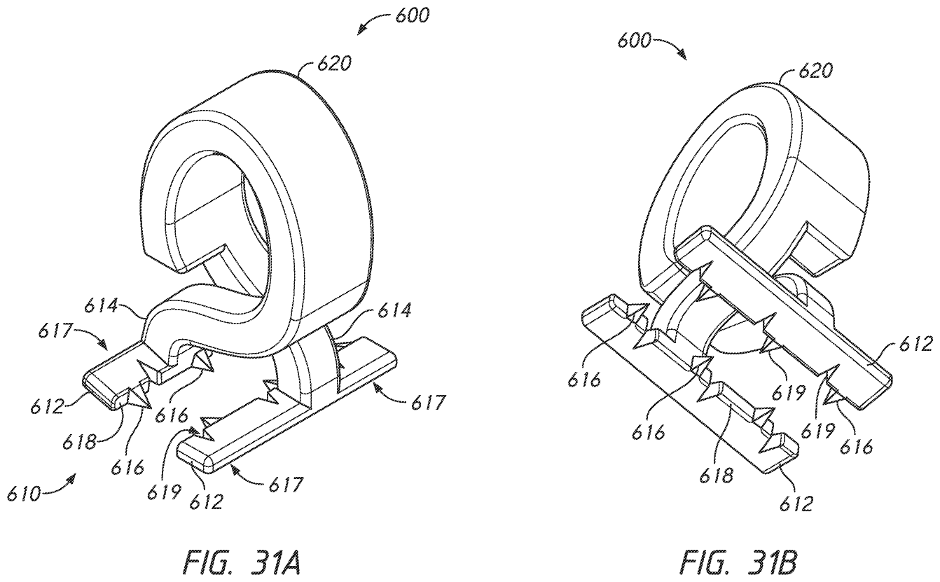

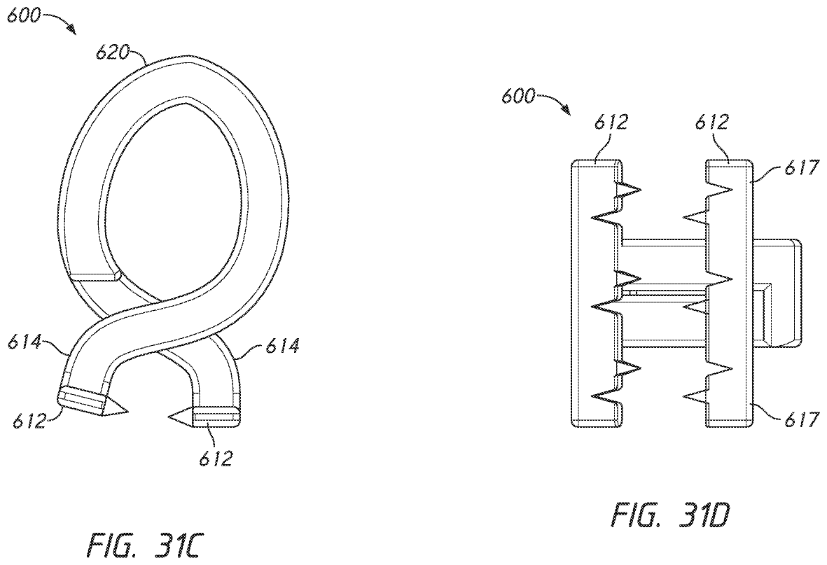

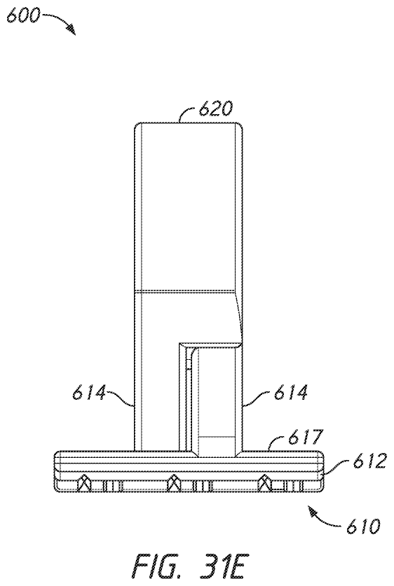

FIGS. 30A-30E show views of a suture clip 500 according to the present disclosure. The suture clip 500 may include one or more of the features of the suture clips of previously discussed examples. For example, the suture clip 500 includes a clamp 510 and a spring 520. The clamp 510 and spring 520 are operatively connected to one another such that the spring 520 applies a closing force urging the jaws 512 of the clamp together. In this example, the jaws of clamp 510 extend substantially at a right angle from the legs 514 toward a midline of the clip 500. As can be observed, e.g., from FIG. 30C, the clip 500 may be substantially symmetric about the midline. The individual jaws 512 of the clamp 510 may be provided as substantially rectangular flat plates 513 that extend perpendicularly from the legs 514. In other embodiments, the jaws may extend toward one another at a different than 90 degree angle. In the illustrated embodiment, the transverse length, which defines the clamping length of the clamp, is greater than a depth dimension of the plate. In other embodiments (e.g., as shown in FIG. 31A), the transverse length of the jaws may be significantly greater (e.g., 3 time, 4 times or more) than the depth dimension D of the jaw. In other embodiments, the plates may be substantially rectangular (i.e., the transverse length and depth may be substantially the same).

The suture clip 500 may include at least one traction element, in this example in the form of a spike 516, extending from one or both of the jaws of the clamp. The traction element(s) may be configured to enable the clip to gain better purchase on the tissue as it applies the closing force thereto. Additionally, the traction element(s) may be arranged on the clamp in a manner which allows the clamp to close more tightly during shape memory training. In this example, the spikes 516 are configured to be slightly offset in opposite directions from a centerline of the jaws 512 and each jaw is provided with a cutout to accommodate the opposing spike 516 in the cutout, thereby allowing the two spikes to nest with one another for a tighter closed state of the clamp. In other embodiments, a different number of suitably arranged spikes and/or other traction elements may be used. To provide a relatively compact form factor, the legs connecting the ends of the spring to the clamp may be offset in opposite transverse directions from the centerline of the clamp, which may produce a moment couple. The traction elements (e.g., spikes) may advantageously also assist in reducing the risk of slip between the clamping surface that may result from any such moment couple. Optionally, one or more surfaces of the jaws, including or other the clamping surfaces from which the spike project (e.g., bottom surfaces 515), may be textured for an enhanced engagement of the clamp with slippery biological tissue.

The suture clip 500 is shown in its neutral state (also referred to as unloaded or closed state) in which the jaws 512 of the clamp 510 are in the closed position. The suture clip 500 may be provided in a loaded (or opened) state by applying a loading (or opening) force, e.g., by squeezing the sides 532 and 534, manually or with a suitable applier tool, to cause the jaws 512 of the clamp to separate. The stiffness of the spring may be selected such that the spring is stiff enough to provide a sufficient closing force to hold the two portions of tissue together without being too still to be opened and or so stiff as to inflicting trauma on the tissue (e.g., punching through the tissue). The suture clip 500 may be formed as a unitary body from a material suitable for use with biological tissue (e.g., a biocompatible metal, a shape memory alloy, a super alloy such as the Cobalt-Chromium-Nickel-Molybdenum alloy sold under the brand name ELGILOY, or other suitable corrosion or oxidation resistant alloy with suitable strength and other mechanical properties). The spring 520 may be formed by shaping a strip of the suitable material into a looped portion 524. Once shaped, the looped portion 524, by virtue of the resilience of the material used, may tend to resist deformation that increase its curvature thus providing a counter force against such deformation. This counter force acts as the spring force of the suture clip 500. The spring force may be enhanced, as described, by the use of a shape memory material, which may be trained to remember the closed position as the memorized/original position, thus providing an even stronger closing force against the tissue.

FIGS. 31A-31E show views of another suture clip 600, which similarly includes a clamp 610 and a spring 620. In this example, the individual jaws 612 extend transversely outward from the legs 614 giving the clip a T-shape when viewed from the side (FIG. 31 E). The clip 600 may thus be interchangeably referred to as T-clip 600. As in other examples here, the clamp 610 of T-clip 600 includes at least one traction element, in this case 3 pyramid-shaped spikes 616 extending from each of the two clamping surface 618. Similar to the clip 500, the T-clip 600 includes a corresponding number of cavities 619 formed in each of the respective clamping surfaces 618 to receive and accommodate substantially fully therein the spikes 616 extending from the opposing clamping surface. In some examples, the spikes may be centrally located, similar to clip 500, or they may be arranged along any portion of the transverse length of the jaw including the transversely extending portions 617. As previously described, the T-clip 600 may be formed of any suitable material such as a biocompatible metal, a shape memory alloy, a super alloy such as the Cobalt-Chromium-Nickel-Molybdenum alloy sold under the brand name ELGILOY, or other suitable corrosion or oxidation resistant alloy with suitable strength and other mechanical properties. In examples in which the T-clip 600 is formed at least in part of a shape memory alloy (for example, at least the spring being formed of a shape memory alloy), the nesting spikes may facilitate an improved shape memory training by providing a tighter closed shape to be memorized by the material. It will be appreciated that in use, the clip 600 may or may not fully close to its nominal closed state, as the spikes may or may not pierce the tissue (e.g., depending on the spring force and type of tissue with which the clip is utilized) when the clamping force is applied. The T-clip 600 is shown in an opened state to better visualize the traction elements of this example. The traction elements, which as described are implemented as pyramid shaped spikes in this specific example, may, in other examples, have a different shape such as conically shaped spikes, triangular pyramid spikes, or other.

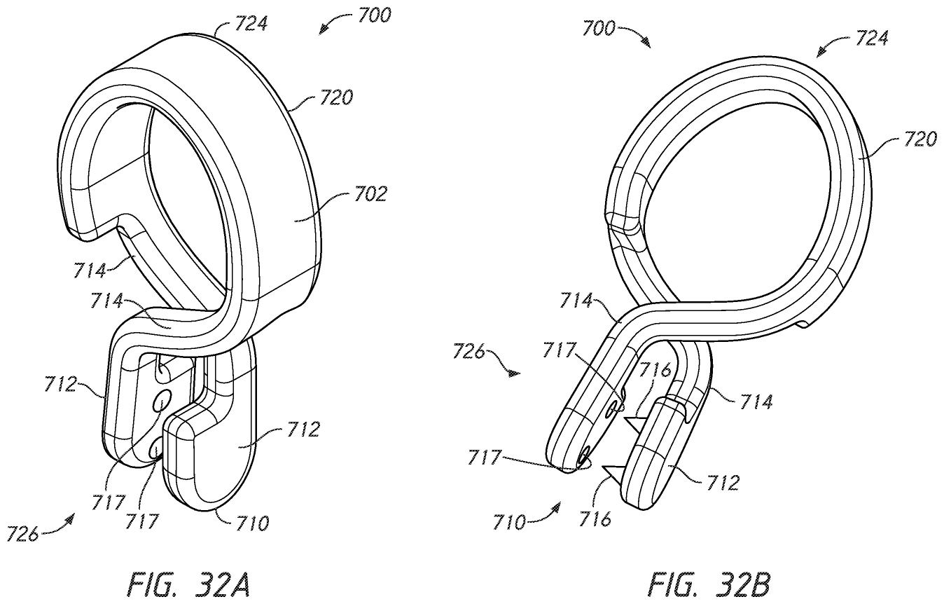

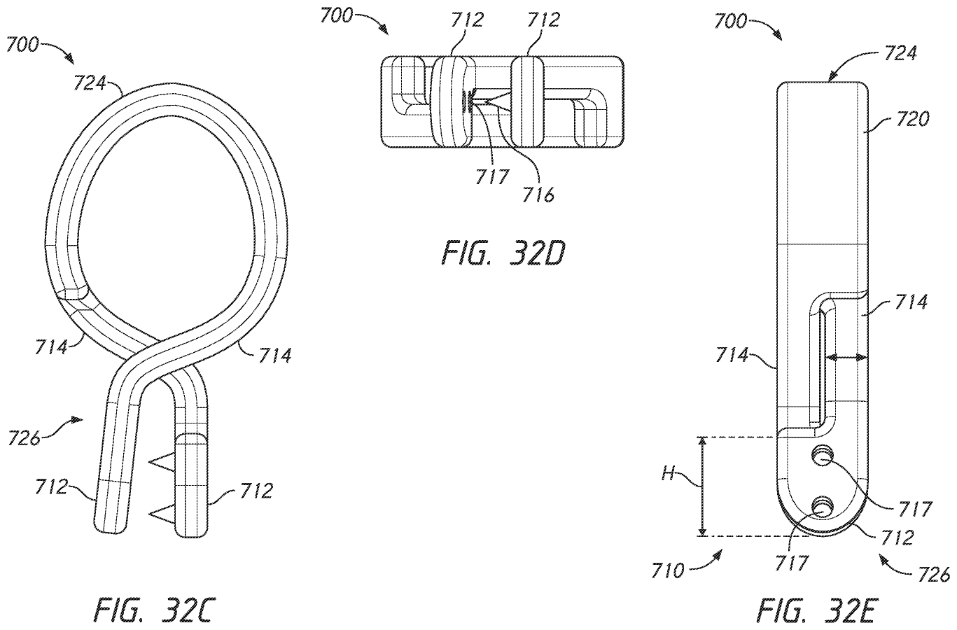

FIGS. 32A-32E show views of suture clip 700 in accordance with the principles of the present disclosure. Similar to other examples, the suture clip 700 may be formed from a unitary strip 702 of material (e.g., shape memory alloy, supper alloy, or another biocompatible metal) which is shaped (e.g. looped) to form a looped upper portion 724 which includes the spring element 720, and a substantially straight lower portion 726 which includes the clamp 710. The looped upper portion 724 may have substantially the same transverse cross section along the length of the spring element 720, which at its opposite ends narrows (abruptly, as shown, or gradually in a tapered manner) to the narrower leg portions that connect the respective ends of the spring element to respective ones of the jaws 712 of clamp 710. In the illustrated example, the width of each of the leg portions 714 is about half the width of the spring element 720 to allow the leg portions to be arranged next to one another and fit within the width footprint of the clip 700. Thus, when viewed from the top, side (FIG. 32E) or bottom (FIG. 32D), the clip 700 has a compact form factor, which may allow for more tightly packing or stacking the clips, such as in a shipping/storage container, which in some cases may be a multi-clip dispensing cartridge. Despite the relatively narrow form factor, clip 700 may provide a sufficiently large clamping surface by the jaws being shaped substantially like paddles. The clip 700 may thus be also interchangeably referred to as paddle clip 700.

The jaws 712 may be tailored to provide the appropriate clamping surface area for apply sufficient clamping force for a given surgical application, e.g., by varying a height H of the paddles. Similar to suture clips of other examples, the clip 700 includes traction elements (e.g., spikes 716, which in the illustrated example are cone-shaped). The spikes 716 are spaced along the height of one of the paddles, extending from the clamping surface of that paddle towards the clamping surface of the other paddle, which includes corresponding number of apertures 717 configured to receive the spikes substantially fully therewithin, when the clamp is in the nominal (unloaded) state. As previously described, providing receiving feature(s) that allow the traction element(s) to be substantially fully contained therein enable a much tighter closed state, which can improve the training of a “remembered” shape of a shape memory alloy. As will be appreciated, the clip 700 is shown in FIGS. 32A-32E in an open state to better illustrate the features of the clamp 710. In the nominal (unloaded) state, the spikes 716 may be received, partially or preferably fully in the apertures 717 to allow the clamping surface to abut one another. Depending on the thickness of the strip of material, which may be configured for achieving a desired spring constant, and the length of the spikes, the apertures may be through apertures extending to the surface opposite the clamping surface (as shown in FIG. 32E), or the apertures may be defined as a cavity formed in the clamping surface and terminating at a depth smaller than the thickness of the strip of material.

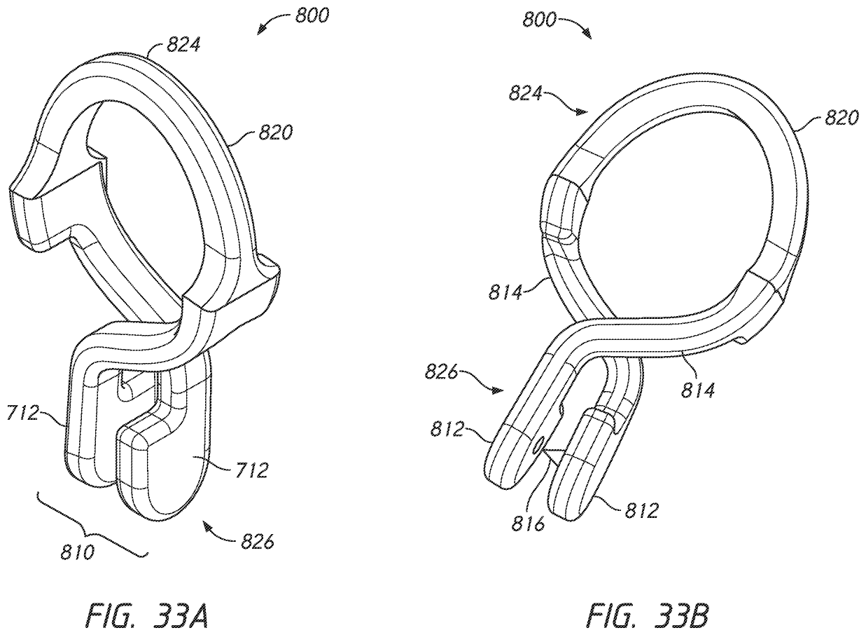

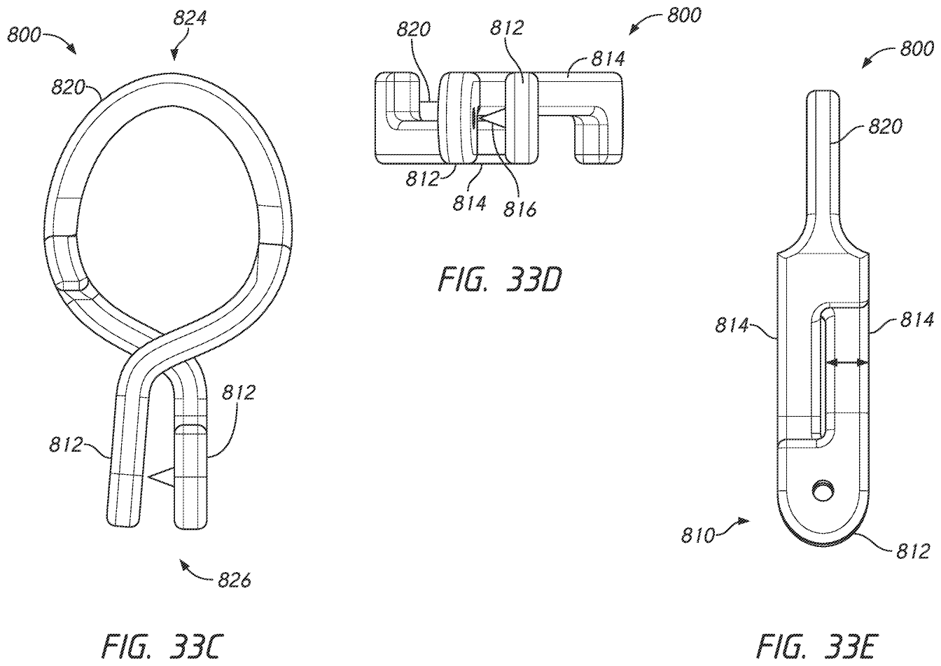

FIGS. 33A-33D shows a further example of a suture clip 800 with paddle-shaped jaws. Like the clip 700 the suture clip 800 includes a spring element 820 as part of the upper looped portion 824, and a clamp 810 as part of the generally straight lower portion 826. This example of a paddle clip differs from the paddle clip 700 in that the spring force provided by spring element 820 has been tailored by tailoring the shape of the looped upper portion. As illustrated, a softer spring may be obtained from essentially a same starting strip of material as in the example if FIG. 32A by reducing the transverse dimension of the strip of material along some or all of the length of the spring element 820. Similar to the suture clip 700, the clip 800 includes connectors or legs 814 extending from the opposite ends of the spring element 820. Similar to paddle clip 700, the clip 800 includes two paddle-shaped jaws 712 coupled to the end of a connector 714. In this illustrated example, the connectors are coupled to the paddle-shaped jaws 712 at a location offset in opposite directions from the centerline of the clamp 710. In other embodiments, a different arrangement may be used. For example a first connector may extend from one end of the spring element to the first jaw substantially along the centerline of the clamp. The other jaw may be connected to the other end of the spring via a pair of second connectors arranged such that they are on opposite sides of the first connector, allowing the first connector to pass therebetween, thus still maintaining a compact form factor of the suture clip. The suture clip 800 in this example has a single traction element (e.g., spike 816) substantially centered on the clamping surface of the paddle. In other embodiments, multiple traction elements may be provided on one or both of the clamping surface, which may be spaced along the height or width dimension or arranged in any other suitable pattern (e.g., a circular pattern, or a rectrangular pattern, with a single spike proximate each corner of a square or rectangular paddle).

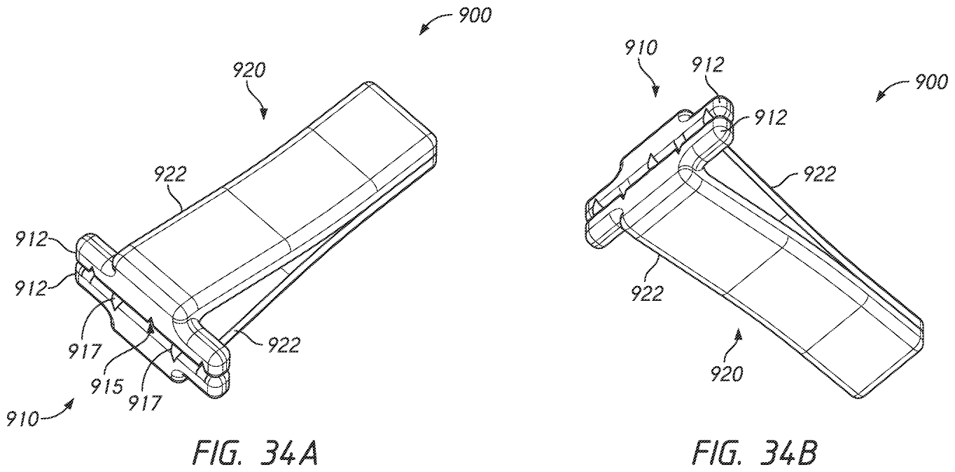

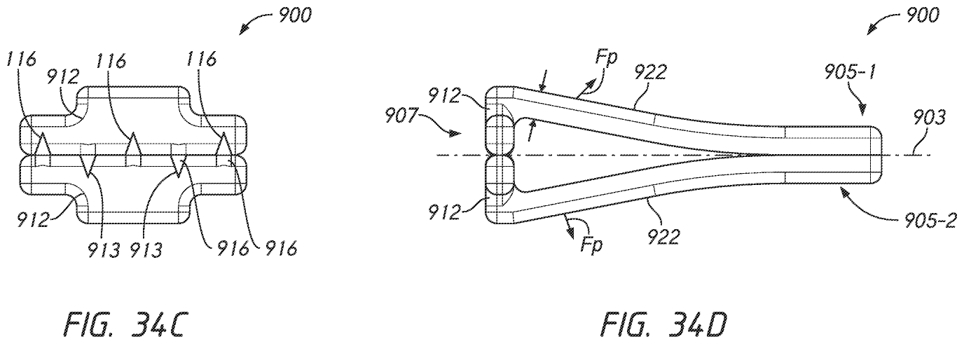

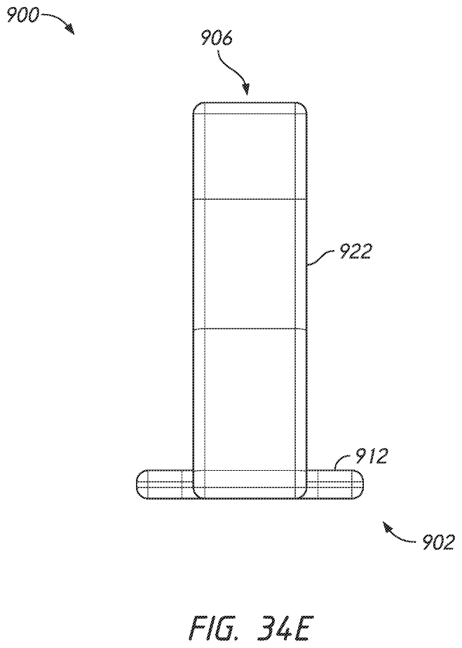

FIGS. 34A-34E show views of a suture clip 900 which has a generally v-shaped configuration and may thus be interchangeably referred to as v-clip 900. Similar to other suture clips described herein, the v-clip 900 includes a clamp 910 and a spring 920. The clamp 910 includes a pair of jaws 912, which extend toward one another, at an angle to the arms 922 of the spring 920. Each of the jaws 912 may be coupled (e.g. by welding) to the respective spring arm 922 or preferably, the jaws 912 may be integrally formed (i.e. as a unitary piece) with the spring arms, such as by bending a distal end 907 of each spring arm toward the midline 903 of the clip 900. In an example manufacturing sequence, each half 905-1 and 905-2 of the v-clip 900 is formed separately and the two halves are joined to form v-clip 900, which as illustrated is symmetric about the midline plane. The individual halves may be formed from two individual strips of suitable material which are joined at the proximal end 906, before or after shaping the strips to form the clamp end of the suture clip 900. Any suitable metal joining techniques (e.g., laser beam welding, friction stir welding, or other suitable welding technique) may be used to join the proximal ends of the strips together. The joining may occur before or after the distal ends of the strips are bent to from the jaws. Similar to other examples, the v-clip 900 includes at least one traction element, in this case a first plurality 911 of spikes 916, extending from one of the jaws, and received in corresponding indentations 915 in the opposite jaw, and a second different plurality 913 of spikes 916 extending form the other jaws and received in corresponding indentations 917 in the first jaw. Unlike some of the previous examples (e.g., suture clips 500, 600, and others) in which the clamp is opened by applying a squeezing force, the clamp 910 of v-clip 900 is configured to be opened by applying a prying force, F_{P} (see e.g., FIG. 34D). The prying force may be applied by a suitable applier tool, such as a tool having an actuation member that inserts between the spring arms, advancing proximally between the spring arms to spread the two spring arms apart from one another, or my manual actuation.

Suture clips of various suitable dimensions may be implemented for various applications. For example, for closure of a Dural incision, a suture clip having any of the exemplary dimensions shown in FIGS. 30C-30E, 31C-31E, 32C-32E, 33C-33E, and 34C-34C may be used. The specific dimensions are illustrative only and may be varied in other embodiments as may be appropriate for a particular surgical application.

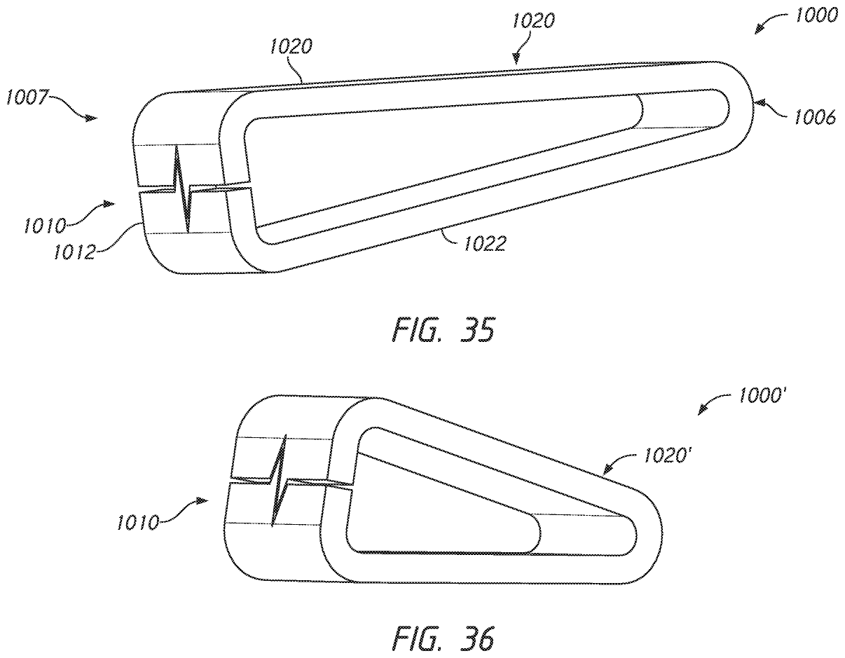

FIGS. 35 and 36 show yet two more examples of v-shaped suture clips according to the present disclosure. The v-clips 1000 and 1000′ have substantially similar configurations to one another, except the overall length of the spring 1020 of the clip 1000 is different from that of the other clip 1000′ so as to tailor the spring force and/or to configure the clips for different medical applications. As shown, the suture clip 1000′ has a relatively shorter spring 1020′ as compared to the spring 1020, making it more compact and thus more suitable for certain surgical applications. Similar to other suture clips described herein, the v-clips 1000 and 1000′ have a spring 1020, 1020′ respectively, and a clamp 1010. For example, referring to v-clip 1000, the spring 1020 has a pair of opposing spring arms 1022, which are joined (e.g., at a bend) at the proximal end 1006 of the clip 1000, and which terminate at the distal end 1007 at the jaws 1012, which form the clamp 1010. The clamp 1010 includes at least one traction element, in this case a pair of opposing spikes 1016 which are arranged to nest in a similar fashion as in the example in FIG. 30A. Like other examples (e.g., as shown and described with respect to FIGS. 11A, 30A and others), the clip 1010 may be similarly formed from a single strip of suitable material which is shaped, in this case bend at a first location near a midpoint along the length of the strip to define the proximal bend, and then at two location near the opposite ends or the strip to define the jaws 1012. The shaping of the strip may be done in any suitable manner for shape memory alloy training, e.g., to allow a shape memory material, if used, to memorize the nominal (closed) shape of the clip so as to augment any natural spring force of the clip urging the clip 1000 closed. The clip 1000′ is similar in configuration, function and form to the clip 1000 with the key difference being the overall length and/or spring force provided by the clip.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

What is claimed is:

1. A suture clip comprising:
a spring element configured to provide a clamping force urging the clip toward a closed configuration;
opposing first and second side portions connected to the spring element such that a manipulation of the first and second side portions toward one another applies a force against the clamping force of the spring element;
a clamp portion comprising:
first and second closure elements coupled to the spring element such that the closure elements are urged toward one another by the clamping force of the spring element;
first and second clamping surfaces provided by the first and second closure elements, respectively, the first and second clamping surfaces arranged opposite one another to apply the camping force to soft tissue positioned between the first and second closure elements; and
at least one spike extending from one of the first and second closure elements toward the other one of the first and second closure elements, the at least one spike configured to penetrate the soft tissue sufficiently to prevent movement of the clamping surfaces relative to the soft tissue while the clamping force is being applied to the tissue by the first and second closure elements.

2. The suture clip of claim 1, wherein the first and second clamping surfaces each have a length, which is at least 5 times greater than a width of the respective clamping surface.

3. The suture clip of claim 1, wherein the spring element, the opposing side portions, and the clamp portion are formed from a unitary piece of material.

4. The suture clip of claim 3, wherein the material is selected from 316 stainless steel, titanium, nickel titanium alloy, and cobalt-chromium-nickel-molybdenum alloy.

5. The suture clip of claim 3, wherein the unitary piece of material is a unitary strip of material having a length, a height, and a thickness, and wherein the thickness and the height of the strip define a width and a length, respectively, of the first and second clamping surfaces.

6. The suture clip of claim 3, wherein the unitary piece of material is a unitary strip of material having a length, a height, and a thickness, and wherein the first and second clamping surfaces are provided by end portions of a side of the strip defined by the length and the height of the strip.

7. The suture clip of claim 6, wherein the height of the strip varies along the length of the strip.

8. The suture clip of claim 1, wherein the first and second closure elements define first and second clamping footprints, and wherein the first and second clamping surfaces each span only a portion of the respective clamping footprint.

9. The suture clip of claim 8, wherein the first and second clamping surfaces are each defined by contact points arranged along at least a portion of a perimeter of the respective clamping footprint.

10. The suture clip of claim 9, wherein the first and second clamping surfaces extend only along the perimeter of the respective clamping footprint.

11. The suture clip of claim 1, wherein at least a portion of the clip is formed of a shape-memory alloy.

12. The suture clip of claim 1, wherein the spring element is configured to apply at least 0.5N of clamping force.

13. The suture clip of claim 1, wherein the first and second closure elements are coupled to the spring element via respective ones of the opposing side portions.

14. The suture clip of claim 1, wherein the spring element comprises a curved piece of surgical-grade metal, the loop having a first end connected to the first side portion and a second end connected to the second side portion such that a convex side of the curved piece is oriented toward the closure elements

15. The clip of claim 1, wherein the spring element has a rectangular transverse cross section.

16. A suture clip comprising a clip body made from a strip of metal, wherein the strip of metal is formed into a closed loop shape having first and second lobe ends spaced from one another, and a middle portion connecting the first and second lobe ends, wherein the middle portion includes a spring side and clamp side, the spring side configured to apply a biasing force to urge the first and second lobe ends away from one another, and the clamp side defining a gap between opposite ends of the strip of material such that the opposite ends can be spaced apart responsive to application of a loading force against the biasing force to allow the clip to be provided in an open configuration, and wherein the clamp side further comprises at least one spike at the gap configured to at least partially penetrate soft tissue positioned between the opposite ends.

17. The suture clip of claim 16, wherein the opposite ends of the strip each have a complimentary shape configured to intermesh with one another when the strip is formed into the closed loop shape.

18. The suture clip of claim 16, wherein the opposite ends are shaped in a complimentary zig-zag pattern defining at least one pair of opposing spikes.

19. A suture clip comprising a spring element and a clamp portion, wherein the spring element is operatively connected to the clamp portion to apply a biasing force urging the clamp portion closed, and wherein the clamp portion includes a pair of opposing surfaces configured to transfer the biasing force to soft tissue to clamp the soft tissue and at least one spike configured to at least partially penetrate the soft tissue to gain purchase on the soft tissue while clamping the soft tissue.

20. A suture clip applier for manipulating a suture clip having a camp portion, opposing actuation sides, and a spring biasing the clamp portion closed, the applier comprising:
a working tip at a distal end of the applier, the working tip comprising:
opposing holding portions configured to engage respective ones of the opposing actuation sides of the clip for holding and opening the clip; and
a stabilizing portion positioned between the holding portions such that the stabilizing portion contacts the spring when the clip is held at the working tip; and
a handle operatively connected to the working tip such that operation of the handle causes one of the holding portions to move toward the other one of the holding portions thereby squeezing the actuation sides together to open the clip, the stabilizing portion remaining in contact with the spring during opening of the clip.
